(19) United States
(12) Patent Application Publication
(10) Pub. No.: US 2022/0235345 A1
(43) Pub. Date: Jul. 28, 2022

(54) METHODS FOR PREPARATION OF ACTIVE SEPARASE

(71) Applicant: University of CA Office of the President, Oakland, CA (US)

(72) Inventors: Laura E. Rosen, Oakland, CA (US); David O. Morgan, Oakland, CA (US)

(73) Assignee: Regents of the University of California, Oakland, CA (US)

(21) Appl. No.: 17/622,318

(22) PCT Filed: Jun. 24, 2020
(86) PCT No.: PCT/US2020/039264

§ 371 Date: Dec. 23, 2021

Related U.S. Application Data
(60) Provisional application No. 62/865,611, filed on Jun. 24, 2019.

(51) Int. Cl. C12N 9/64; C07K 14/47

(52) U.S. Cl. CPC C12N 9/6472; C07K 2319/50; C12Y 304/22049; C07K 14/47

(57) ABSTRACT

Provided herein are polypeptide constructs containing a securin, e.g., a full-length securin or a truncated securin, fused to a separase. The polypeptide constructs may further contain linker peptides, protease recognition sites, and unfoldase recognition sites to facilitate expression and/or purification. Methods for obtaining the polypeptide constructs with active separase activity are also described, as well as methods for identify separase modulator compounds such as separase inhibitors.

### CROSS-REFERENCE TO RELATED PATENT APPLICATIONS

The present patent application claims benefit of priority to U.S. Provisional Patent Application No. 62/865,611, filed Jun. 24, 2019, which is incorporated by reference for all purposes.

### STATEMENT AS TO RIGHTS TO INVENTIONS MADE UNDER FEDERALLY SPONSORED RESEARCH AND DEVELOPMENT

This invention was made with government support under Grant No. R35 GM118053 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Jun. 5, 2020, is named 081906-1191728-231610WO_SL.txt and is 176,274 bytes in size.

### BACKGROUND OF THE INVENTION

The protease separase initiates chromosome segregation in anaphase by cleaving the kleisin subunit (Scc1/Rad21) of the cohesin protein complex, allowing the duplicated eukaryotic chromosomes to be segregated to opposite poles of the cell¹⁻³. Tight regulation of separase function is critical, as premature cleavage of cohesin can lead to chromosome loss and genomic instability.

Separase is a large caspase-family cysteine protease (the human protein is 2,120 amino acids/233 kDa). Approximately one quarter of human separase is comprised of the C-terminal protease domain, which is conserved across eukaryotes and of which it is possible to make a structural model based on homology to orthologous structures⁴⁻⁷. The large N-terminal region is poorly conserved and there is currently no detailed structural model of this region in the human protein, although it is likely composed of superhelical repeats like those seen in structures of separase from budding yeast⁵ and C. elegans⁶. Between the helical N-terminal region and the C-terminal protease domain, human separase contains regions that are predicted to be intrinsically disordered.

Separase cleavage sites have a consensus motif of ExxR, with cleavage occurring after the arginine^{1,2,8}. An acidic or phosphorylated residue immediately upstream of the ExxR promotes cleavage^{4,8-10}. In the structure of the separase protease domain from C. thermophilum, basic and acidic binding pockets accommodate, respectively, the glutamate and arginine of the consensus motif⁴. Two ExxR sites are thought to be cleaved in the human Scc1 substrate⁸. Human separase contains four ExxR sites in its central disordered region, three of which are subjected to autocleavage upon separase activation“. After autocleavage, the N- and C-terminal domains of separase remain bound, with no apparent loss of protease activity”. C. elegans separase has shorter but similarly located intrinsically disordered regions, and its structure reveals that association of the N- and C-terminal domains does not depend on the disordered polypeptide chain between them⁶.

In early mitosis, separase is inhibited by a high-affinity interaction with the protein securin. Securin is thought to be intrinsically disordered when free in solution¹², and the structures of securin-separase complexes from budding yeast⁵ and C. elegans⁶ reveal that securin binds as an extended polypeptide along the length of separase. A pseudosubstrate motif on securin interacts with the active site⁴, presumably blocking substrate interactions. Securin inhibition is relieved when the N-terminal region of securin is ubiquitinated by the APC/C in metaphase, targeting it for destruction by the proteasome. Other vertebrate-specific modes of separase regulation have been identified, including inhibition by cyclin B-Cdk1 binding to separase in a manner dependent on proline isomerization by Pin1¹³, but the specific molecular mechanism for this inhibition remains unknown.

The ExxR separase cleavage motif is ubiquitous in the proteome, but very few of these motifs are known to be cleaved by separase. Human Scc1 contains six ExxR motifs, for example, but only two are cleaved in mitosis⁸. Therefore, it seems likely that there are other as yet unidentified mechanisms governing separase activity at the substrate level. Many proteases contain exosites: protease regions distinct from the active site that bind substrate sequences away from the cleavage site, thereby enhancing reaction efficiency¹⁴. The only evidence for separase regulation by substrate engagement outside of the cleavage site is that the securin-separase complex binds to DNA, helping to localize it to chromosomes¹⁵. While this binding results in increased cleavage of DNA-associated substrates, DNA does not enhance the enzyme's catalytic rate, and this interaction is too general to explain the observed specificity of separase.

Separase was identified two decades ago^{1,2,16} and its central role in cell division is well established. However, many basic questions about its biochemical behavior and regulation remain unanswered, in part because of the difficulty of producing active protein amenable for biochemical and biophysical studies. It is well established that soluble separase can only be obtained in recombinant systems by co-expression with securin, as securin appears to be a co-translational separase-folding chaperone in addition to being an inhibitory. Therefore, production of active separase typically begins with purification of the securin-separase complex, from which securin is removed using the APC/C-proteasome system (for human separase, an incubation with Xenopus egg extract serves this purpose)^{13,18-20}. While this protocol is sufficient for certain experiments, it does not produce the quantities and purity of protein needed for detailed biophysical studies.

### BRIEF SUMMARY OF THE INVENTION

Provided herein are polypeptide constructs containing a securin fused to a separase. In some embodiments the securin is a full-length securin. In some embodiments, the securin is a truncated securin. Polypeptide constructs containing a securin linked to an unfoldase recognition site are also provided.

Also provided herein are methods for identifying a separase modulator compound. The methods include:

(i) measuring a level or rate of peptide substrate cleavage by a polypeptide construct in the presence of a candidate compound, wherein the polypeptide construct comprises a securin fused to a separase;

(ii) measuring a level or rate of peptide substrate cleavage by the polypeptide construct in the absence of the candidate compound; and

(iii) identifying the candidate compound as a separase modulator compound when the level or rate of peptide substrate cleavage in step (i) is higher or lower than the level or rate of peptide substrate cleavage in step (ii). In some embodiments, the peptide substrate comprises an LPE motif.

Also provided herein are methods for obtaining an active separase. In some embodiments, the methods include:

(a) co-expressing a separase and a securin, wherein the securin is linked to an unfoldase recognition site; and

(b) combining the co-expressed separase and securin with an unfoldase-peptidase complex;

thereby removing the securin and obtaining the active separase.

In some embodiments, the methods for obtaining an active separase include:

(1) expressing a polypeptide comprising a securin fused to a separase; and

(2) removing the securin from the expressed polypeptide, thereby obtaining the active separase; wherein the active separase is substantially free of the securin.

### BRIEF DESCRIPTION OF THE DRAWINGS

FIG. 1A shows a cartoon diagram of the securin-separase complex. The vertical dashed line indicates an approximate delineation between the separase N-terminal helical domain and C-terminal protease domain containing the active site. The C-terminal region of securin binds in an antiparallel fashion along the length of separase, and begins with the pseudosubstrate motif bound in the separase active site. The N-terminal region of securin contains the APC/C degrons.

FIG. 1B shows a diagram of the securin-separase fusion construct. Also depicted are the flexible Gly-Ser linker separating securin and separase and the regions of human separase predicted to be intrinsically disordered (IDR).

FIG. 1C shows the analysis of purified securin-separase fusion protein by SDS-PAGE and stained with Coomassie Blue, using molecular weight markers as indicated.

FIG. 1D shows the analysis of purified securin-separase (top) and apo (active) separase (bottom) by negative-stain EM. Five representative class averages of each preparation are shown.

FIG. 1E shows the evaluation of securin-separase binding to fluorescein-labeled DNA by fluorescence polarization. Two 50 bp dsDNA molecules with the same base composition but different sequence were tested, as well as a 25 bp molecule. Data points indicate means (+/− SEM) of triplicate samples.

FIG. 1F shows a schematic of separase activation by the ubiquitin-proteasome system, whereby securin is tagged for degradation and removed, which can be recapitulated using an N-terminal ClpX-specific sequence and the bacterial protease ClpXP.

FIG. 1G shows the analysis of Scc1 fragment cleavage. Securin-separase fusion protein was incubated with TEV protease, ATP, and/or the ClpXP ATPase as indicated, and separase activity was measured by cleavage of an ³⁵S-labeled Scc1 fragment (residues 142-300) produced by translation in vitro.

FIG. 1H shows a plot of initial velocity vs. peptide concentration. Michaelis-Menten analysis was performed with purified, active separase and the peptide DDREIMREGS (SEQ ID NO: 25), which includes cleavage site 1 in Scc1. The peptide sequence was flanked by the MCA fluorophore and DNP quencher, and cleavage was monitored by an increase in fluorescence. Initial velocity was normalized to enzyme concentration. Data points indicate means (+/− SEM) of triplicate samples.

FIG. 2A shows a diagram of the Scc1 sequence, including the locations of two separase cleavage sites, LPE motif, and boundaries of truncated constructs evaluated in FIG. 2B and FIG. 2C. Figure discloses SEQ ID NOS 27 and 28, respectively, in order of appearance.

FIG. 2B shows the reaction products resulting from ³⁵S-labeled Scc1 fragments incubated with active or inactive separase as indicated, as analyzed by SDS-PAGE and Phosphorimaging.

FIG. 2C shows the reaction products resulting from separase incubated with an ³⁵S-labeled Scc1 fragment (aa 142-300) in which the indicated residues were changed to alanines, as analyzed by SDS-PAGE and Phosphorimaging. The sequence of the relevant region of Scc1 is shown (SEQ ID NO: 29).

FIG. 3A shows a schematic of the separase biosensor used to evaluate cleavage in vivo, which includes histone H2B, red fluorescent protein (RFP), the indicated Scc1 fragment, and green fluorescent protein (GFP)²⁸.

FIG. 3B shows the time course of wild-type (WT) biosensor cleavage by separase, showing green fluorescence (left), red fluorescence (center), and merged images (right). Time zero is the last time point before the onset of chromosome segregation. Biosensor cleavage is indicated by reduced green fluorescence relative to red fluorescence.

FIG. 3C shows representative images demonstrating late anaphase fluorescence of biosensor variants carrying mutations in Scc1 (WT, wild-type; NC, non-cleavable mutations at sites 1 and 2; LP→AA, mutations of ²⁵⁵LP; Δ10aa, deletion of aa 251 to 260, which contain the LPE motif).

FIG. 3D shows the quantification of the loss of GFP fluorescence in the four biosensor variants shown in FIG. 3C. Data points indicate means (+/− SEM) from between 15 and 30 cells.

FIG. 4A shows a cartoon diagram of the securin-separase fusion protein containing the full separase-binding region of securin (left) or with securin truncated on the C-terminal side of the pseudosubstrate motif (right). The separase active site (circle) and pseudosubstrate motif (rectangle overlapping circle at left) are indicated.

FIG. 4B shows a diagram of the human securin sequence, indicating the locations of the pseudosubstrate sequence (EIEKFFP (SEQ ID NO: 26)), all LP sites including the ¹³⁰LPE motif, and the positions of the three truncations tested.

FIG. 4C shows a plot generated for Michaelis-Menten analysis performed with the three indicated securinΔ-separase fusion proteins, compared with purified separase lacking securin. Initial velocity was normalized by enzyme concentration. Data points indicate means (+/− SEM) of triplicate samples.

FIG. 4D shows the reaction products resulting from incubation of ³⁵S-labeled Scc1 fragments (aa 142-300), with or without mutations in the ²⁵⁵LPE motif, with the three indicated securinΔ-separase fusion proteins or with purified separase lacking securin, as analyzed by SDS-PAGE and Phosphorimaging.

FIG. 4E shows that the pseudosubstrate motif in securin was converted to a separase cleavage site using two point mutations (¹¹⁸FP to RE). Separase was incubated with an ³⁵S-labeled securin fragment (aa 93-150) containing these mutations as well as mutations in the indicated LP motifs. Reaction products were analyzed by SDS-PAGE and Phosphorimaging.

FIG. 4F shows sequence alignment of securin pseudosubstrate motifs (EIE, DIE, or EVE at left), indicating the downstream conserved LPE motifs (highlighted at right) (SEQ ID NOS 30-37, respectively, in order of appearance).

FIG. 4G shows a cartoon diagram of the securin-separase complex, illustrating the pseudosubstrate motif interaction with the active site and the LPE motif interaction with the separase exosite.

### DETAILED DESCRIPTION OF THE INVENTION

The present invention was developed with the use of protein engineering for the generation of active separase. Using this active separase protein, it was discovered that rapid cleavage of Scc1 requires a sequence motif in Scc1 that is distinct from the cleavage motif, and which interacts with a docking site (exosite) on separase. It is demonstrated herein that securin binding interferes with separase engagement of the substrate docking motif, identifying a second mechanism by which securin inhibits cohesin cleavage by separase. The methods and polypeptide constructed provided herein allow for the production of large amounts of homogeneous, fully active enzyme for a variety of studies.

### I. POLYPEPTIDE CONSTRUCTS

Provided herein are polypeptide constructs comprising a securin fused to a separase. As noted above, separase is a cysteine protease containing a large superhelical N-terminal region and a conserved C-terminal protease domain^{4, 5, 6, 21} The protease domain includes a substrate binding domain and a caspase-like catalytic domain. The substrate binding domain is characterized by a mixed α/β fold, having a four-helix bundle packed against an RNase H-like β-sheet. This five-stranded, mostly anti-parallel β-sheet also contains a two-helix hairpin extension between strands 3 and 4. The caspase-like catalytic domain contains a central six-stranded, mostly parallel β-sheet flanked by α-helices. Catalytic cysteine and histidine residues are located in the β-sheet in loops following strands 3 and 4, respectively. The large N-terminal region adopts an extended conformation in species such as H. sapiens (UniProt Q14674) and S. cerevisiae, while a closed conformation is adopted in species such as C. elegans. The N-terminal region contains multiple HEAT repeat units (26 in H. sapiens), with each HEAT having a pair of anti-parallel α-helices linked by a flexible loop, and a disordered region between the HEAT repeat units and the C-terminal protease domain. Securin binds in an extended conformation along the length of separase. For example, two short helices are the only secondary structural features observed by X-ray crystallography in the S. cerevisiae securin-separase complex. Residues 258-269 of the S. cerevisiae securin (corresponding to residues 113-224 of H. sapiens securin; UniProt O95997) lie in the separase active site upon formation of the securin-separase complex.

In some embodiments, the separase comprises an amino acid sequence having at least 70% identity (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity) to H. sapiens separase (SEQ ID NO:1), M. musculus separase (SEQ ID NO:7; UniProt P60330), C. elegans separase (SEQ IQ NO:8; UniProt G5ED39), S. cerevisiae separase (SEQ ID NO:9; UniProt Q03018), or S. pombe separase (SEQ ID NO:10; UniProt P18296). In some embodiments, the separase comprises an amino acid sequence having at least 80% identity to H. sapiens separase, M. musculus separase, C. elegans separase, S. cerevisiae separase, or S. pombe separase. In some embodiments, the separase comprises an amino acid sequence having at least 90% identity to H. sapiens separase, M. musculus separase, C. elegans separase, S. cerevisiae separase, or S. pombe separase. In some embodiments, the separase comprises an amino acid sequence having at least 90% identity to SEQ ID NO:1.

Percentage of sequence identity can be determined by comparing two optimally aligned sequences over a comparison window, wherein the portion of the sequence (e.g., a peptide of the invention) in the comparison window may comprise additions or deletions (i.e., gaps) as compared to the reference sequence which does not comprise additions or deletions, for optimal alignment of the two sequences. The percentage can be calculated by determining the number of positions at which the identical amino acid residue occurs in both sequences to yield the number of matched positions, dividing the number of matched positions by the total number of positions in the window of comparison and multiplying the result by 100 to yield the percentage of sequence identity.

“Identical” and “identity,” in the context of two or more polypeptide sequences or nucleic acid sequences, refer to two or more sequences or subsequences that are the same. Sequences are “substantially identical” to each other if they have a specified percentage of nucleotides or amino acid residues that are the same (e.g., at least 70%, at least 75%, at least 80%, at least 85%, at least 90%, at least 95%, or at least 99% identical over a specified region), when compared and aligned for maximum correspondence over a comparison window, or designated region as measured using one of the following sequence comparison algorithms or by manual alignment and visual inspection.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Examples of algorithms that are suitable for determining percent sequence identity and sequence similarity are the BLAST and BLAST 2.0 algorithms, which are described in Altschul et al., (1990) J. Mol. Biol. 215: 403-410 and Altschul et al. (1977) Nucleic Acids Res. 25: 3389-3402, respectively. Software for performing BLAST analyses is publicly available at the National Center for Biotechnology Information website, ncbi.nlm.nih.gov. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see, e.g., Henikoff and Henikoff, Proc. Natl. Acad. Sci. USA 89:10915 (1989)).

The securin may be a full-length securin or a truncated securin. In some embodiments, the full-length securin comprises an amino acid sequence having at least 70% identity to H. sapiens securin (SEQ ID NO:2), M. musculus securin (SEQ ID NO:11; UniProt Q9CQJ7), C. elegans securin (SEQ IQ NO:12; UniProt Q18235), S. cerevisiae securin (SEQ ID NO:13; UniProt P40316), or S. pombe securin (SEQ ID NO:14; UniProt P21135). In some embodiments, the full-length securin comprises an amino acid sequence having at least 80% identity to H. sapiens securin, M. musculus securin, C. elegans securin, S. cerevisiae securin, or S. pombe securin. In some embodiments, the full-length securin comprises an amino acid sequence having at least 90% identity to H. sapiens securin, M. musculus securin, C. elegans securin, S. cerevisiae securin, or S. pombe securin. In some embodiments, the full-length securin comprises an amino acid sequence having at least 90% identity to SEQ ID NO:2.

In some embodiments, the truncated securin contains an amino acid sequence having at least 70% identity to residues 10-202 of SEQ ID NO:2, or a shorter polypeptide corresponding to, e.g., residues 20-202, or 30-202, or 40-202, or 50-202, or 60-202, or 70-202, or 80-202, or 90-202, or 100-202, or 110-202, or 120-202, or 130-202, or 140-202, or 150-202, or 160-202, or 170-202, or 180-202, or 190-202 of SEQ ID NO:2.

In some embodiments, the truncated securin contains an amino acid sequence having at least 70% identity to residues 10-199 of SEQ ID NO:11, or a shorter polypeptide corresponding to, e.g., residues 20-199, or 30-199, or 40-199, or 50-199, or 60-199, or 70-199, or 80-199, or 90-199, or 100-199, or 110-199, or 120-199, or 130-199, or 140-199, or 150-199, or 160-199, or 170-199, or 180-199, or 190-199 of SEQ ID NO:11.

In some embodiments, the truncated securin contains an amino acid sequence having at least 70% identity to residues 10-244 of SEQ ID NO:12, or a shorter polypeptide corresponding to, e.g., residues 20-244, or 30-244, or 40-244, or 50-244, or 60-244, or 70-244, or 80-244, or 90-244, or 100-244, or 110-244, or 120-244, or 130-244, or 140-244, or 150-244, or 160-244, or 170-244, or 180-244, or 190-244 of SEQ ID NO:12.

In some embodiments, the truncated securin contains an amino acid sequence having at least 70% identity to residues 10-373 of SEQ ID NO:13, or a shorter polypeptide corresponding to, e.g., residues 20-373, or 30-373, or 40-373, or 50-373, or 60-373, or 70-373, or 80-373, or 90-373, or 100-373, or 110-373, or 120-373, or 130-373, or 140-373, or 150-373, or 160-373, or 170-373, or 180-373, or 190-373, or 200-373, or 210-373, or 220-373, or 230-373, or 240-373, or 250-373, or 260-373, or 270-373, or 280-373, or 290-373, or 300-373, or 310-373, or 320-373, or 330-373, or 340-373, or 350-373, or 360-373 of SEQ ID NO:13.

In some embodiments, the truncated securin contains an amino acid sequence having at least 70% identity to residues 10-301 of SEQ ID NO:14, or a shorter polypeptide corresponding to, e.g., residues 20-301, or 30-301, or 40-301, or 50-301, or 60-301, or 70-301, or 80-301, or 90-301, or 100-301, or 110-301, or 120-301, or 130-301, or 140-301, or 150-301, or 160-301, or 170-301, or 180-301, or 190-301, or 200-301, or 210-301, or 220-301, or 230-301, or 240-301, or 250-301, or 260-301, or 270-301, or 280-301, or 290-301 of SEQ ID NO:14.

In some embodiments, the securin comprises an amino acid sequence having at least 90% identity to positions 93-202 of SEQ ID NO:2. In some embodiments, the securin consists of an amino acid sequence having at least 90% identity to positions 160-202 of SEQ ID NO:2. In some embodiments, the securin consists of an amino acid sequence having at least 90% identity to positions 138-202 of SEQ ID NO:2. In some embodiments, the securin consists of an amino acid sequence having at least 90% identity to positions 127-202 of SEQ ID NO:2.

In some embodiments, the securin is fused to the separase via a linker, e.g., a linker peptide. As used herein, the term “linker” refers to a peptidic moiety or a non-peptidic moiety that covalently connects one terminus of a securin to one terminus of a separase. In some embodiments, the linker covalently connects the C-terminus of the securin to the N-terminus of the separase. A number of linkers can be used for fusion of the securin to the separase including, for example, rigid, flexible, and cleavage linkers such as those described by Chen et al. (Adv Drug Deliv Rev. 2013; 65(10): 1357-1369). In some embodiments, the linker contains a flexible peptide such as GGGGS (SEQ ID NO:15), (GGGGS)₂ (SEQ ID NO:16), (GGGGS)₃ (SEQ ID NO:17), (GGGGS)₄ (SEQ ID NO:18), GGGGGG (SEQ ID NO:19), GGGGGGGG (SEQ ID NO:20), GGSGGSGGGSGGGSG (SEQ ID NO:21), or the like.

In some embodiments, the polypeptide construct comprises a protease recognition site. A linker peptide in the polypeptide construct, for example, may contain one or more recognition sites for proteases such as those described by Waugh (Protein Expr Purif. 2011; 80(2): 283-293). In some embodiments, the protease is a site-specific endopeptidase. Examples of suitable site-specific endopeptidases include, but are not limited to, FactorXa, enterokinase, α-thrombin, human rhinovirus 3C protease, Tobacco Vein Mottling Virus (TVMV) protease, and Tobacco Etch Virus (TEV) protease. In some embodiments, the protease is TEV protease.

In some embodiments, the polypeptide construct contains one or more affinity tags, e.g., for the purposes of detection or purification. A number of suitable tags can be included in the polypeptide constructs including, for example, those described by Kimple et al. (Curr Protoc Protein Sci. 2013; 73(1): 9.9.1-9.9.23). Examples of affinity tags include, but are not limited to, a calmodulin binding peptide (CBP), a chitin binding domain (CBD), a dihyrofolate reductase (DHFR) moiety, a FLAG epitope, a glutathione S-transferase (GST) tag, a hemagglutinin (HA) tag; a maltose binding protein (MBP) moiety; a Myc epitope; a polyhistidine tag (e.g., HHHHHH, SEQ ID NO: 22); and streptavidin-binding peptides (e.g., those described in U.S. Pat. No. 5,506,121). An affinity tag may be included at one or more locations in the polypeptide construct. An affinity tag such as a streptavidin-binding peptide may reside, for example, at the N-terminus of the polypeptide construct or at the C-terminus of the polypeptide construct. In some embodiments, the linker peptide comprises an affinity tag, e.g., a FLAG epitope containing the sequence DYKDDDDK (SEQ ID NO: 23) with or without additional amino acid residues.

In some embodiments, the polypeptide construct further includes a recognition site for an unfoldase, e.g., an E. coli unfoldase, linked to the securin. E. coli have a collection of energy-dependent proteases that couple ATP hydrolysis to the translocation of a substrate protein to a sequestered proteolytic chamber. These include ClpXP, ClpAP, lon, HslUV, and FtsH. ClpXP is a complex of a hexamer of the ClpX unfoldase and the 14-mer ClpP protease. Upon substrate recognition, ClpX uses the energy from ATP hydrolysis to processively translocate along the substrate polypeptide chain, unfolding the substrate, and delivering the unfolded protein into the lumen of the ClpP structure where it encounters a high concentration of serine protease active sites. In some embodiments, the unfoldase recognition site is an E. coli ClpX recognition site. In some embodiments, the unfoldase recognition site contains the sequence TNTAKILNFGR (SEQ ID NO:24). In some embodiments, the unfoldase recognition site is linked to the securin via an affinity tag, e.g., a streptavidin-binding peptide.

Also provided herein are polypeptide constructs having a securin linked to an unfoldase recognition site. The securin may be linked to the unfoldase recognition site with any of the linkers described herein, and the construct may further contain any of the affinity tags and protease recognition sites described above.

In some embodiments, the polypeptide construct includes an amino acid sequence according to SEQ ID NO:3, SEQ ID NO. 4, SEQ ID NO. 5, or SEQ ID NO. 6. The polypeptide constructs described herein, as well as specific securin portions and/or separase portions therein, can be used with or without N-terminal methionine residues (e.g., with or without the N-terminal methionine residues set forth in SEQ ID NOS:1-14).

### II. COMPOSITIONS AND METHODS FOR IDENTIFYING SEPARASE MODULATORS

The securin-separase fusion constructs described herein can be used to facilitate basic studies of separase enzyme behavior, including its activity toward various substrates. The fusion constructs can be used, for example, as reagents for the mechanistic study of chromosome segregation. In addition, the fusion constructs can be used in the screening of chemical modulators (e.g., separase inhibitors) that may have research or therapeutic potential. Accordingly, some embodiments of the present disclosure provide a mixture comprising a polypeptide construct as described above and one or more test substances. In some embodiments, the test substance is an organic small-molecule separase inhibitor candidate.

Also provided herein are methods for identifying a separase modulator compound. The methods include:

(i) measuring a level or rate of peptide substrate cleavage by a polypeptide construct in the presence of a candidate compound, wherein the polypeptide construct comprises a securin fused to a separase;

(ii) measuring a level or rate of peptide substrate cleavage by the polypeptide construct in the absence of the candidate compound; and

(iii) identifying the candidate compound as a separase modulator compound when the level or rate of peptide substrate cleavage in step (i) is higher or lower than the level or rate of peptide substrate cleavage in step (ii). In some embodiments, the level or rate of peptide substrate cleavage in step (i) is lower than the level or rate of the peptide substrate cleavage in step (ii), and the candidate compound is identified as a separase inhibitor.

In some embodiments, the peptide substrate contains a cohesin Scc1 subunit sequence, such as an Scc1 site 1 sequence containing EIMR (SEQ ID NO: 27) (e.g., DDREIMREGS; SEQ ID NO:25). In addition to the Scc1 sequence, the peptide substrate may further contain a pair of fluorescence resonance energy transfer (FRET) partners to facilitate detection of substrate cleavage as described in more detail below. One non-limiting example of a FRET partner pair, for instance, is an Mca moiety (i.e., (7-methoxycoumarin-4-yl)acetyl) covalently bonded to a first terminus of the peptide substrate and a Dnp moiety (i.e., 2,4-dinitrophenyl) covalently bonded to the second terminus of the peptide substrate). A number of suitable FRET partners and other useful signal-generating moieties are described, for example, by Ong, et al. (Analyst, 2017, 142, 1867-1881). In some embodiments, the peptide substrate comprises an LPE motif (i.e., a leucine-proline-glutamic acid motif), which is present in the cohesin Scc1 subunit and also in the native securin sequence.

### III. METHODS FOR PREPARING/OBTAINING ACTIVE SEPARASE

Also provided herein are methods for obtaining an active separase fusion protein. The methods include expressing a polypeptide comprising a truncated securin fused to a separase (e.g., a polypeptide construct having a sequence as set forth in SEQ ID NOS:4-6), thereby obtaining the active separase fusion protein. The methods may include the use of nucleic acids encoding a polypeptide construct as described above, as well as vectors containing the nucleic acids and host cells containing the nucleic acids and/or the vectors.

Nucleic acids encoding the polypeptide constructs can be obtained using routine techniques in the field of recombinant genetics. Basic texts disclosing such techniques include Sambrook and Russell, Molecular Cloning, A Laboratory Manual (3rd ed. 2001); Kriegler, Gene Transfer and Expression: A Laboratory Manual (1990); and Current Protocols in Molecular Biology (Ausubel et al., eds., 1994-1999). Nucleic acids encoding the polypeptide constructs may also be obtained through in vitro amplification methods such as those described herein and in Berger, Sambrook, and Ausubel, as well as Mullis et al., (1987) U.S. Pat. No. 4,683,202; PCR Protocols A Guide to Methods and Applications (Innis et al., eds) Academic Press Inc. San Diego, Calif. (1990) (Innis); Arnheim & Levinson (Oct. 1, 1990) C&EN 36-47; The Journal Of NIH Research (1991) 3: 81-94; Kwoh et al. (1989) Proc. Natl. Acad. Sci. USA 86: 1173; Guatelli et al. (1990) Proc. Natl. Acad. Sci. USA 87, 1874; Lomell et al. (1989) J. Clin. Chem., 35: 1826; Landegren et al., (1988) Science 241: 1077-1080; Van Brunt (1990) Biotechnology 8: 291-294; Wu and Wallace (1989) Gene 4: 560; and Barringer et al. (1990) Gene 89: 117.

One of skill will recognize that modifications can additionally be made without diminishing the biological activity of the securin or the separase. Some modifications may be made to facilitate the cloning, expression, or incorporation of a domain into a fusion protein. Such modifications include, for example, the addition of codons at either terminus of the polynucleotide that encodes the binding domain to provide, for example, a methionine added at the amino terminus to provide an initiation site, or additional amino acids (e.g., poly His) placed on either terminus to create conveniently located restriction sites or termination codons or purification sequences.

The fusion polypeptides as described herein can be expressed in a variety of host cells, including E. coli, other bacterial hosts, yeasts, filamentous fungi, and various higher eukaryotic cells such as the Sf9, COS, CHO and HeLa cell lines and myeloma cell lines. There are many expression systems for producing the polypeptides that are well known to those of ordinary skill in the art. (See, e.g., Gene Expression Systems, Fernandex and Hoeffler, Eds. Academic Press, 1999; Sambrook and Russell, supra; and Ausubel et al., supra.) Typically, a polynucleotide that encodes the polypeptide is placed under the control of a promoter that is functional in the desired host cell. Many different promoters are available and known to one of skill in the art, and can be used in the expression vectors of the invention, depending on the particular application. Ordinarily, the promoter selected depends upon the cell in which the promoter is to be active. Other expression control sequences such as ribosome binding sites, transcription termination sites and the like are also optionally included. Constructs that include one or more of these control sequences are termed “expression cassettes.”

Eukaryotic expression systems for producing the polypeptide constructs—including insect cells, yeast, and mammalian cells—are well known in the art and are also commercially available. Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the CMV promoter, SV40 early promoter, SV40 later promoter, metallothionein promoter, murine mammary tumor virus promoter, Rous sarcoma virus promoter, polyhedrin promoter, p10 promoter, or other promoters shown effective for expression in eukaryotic cells.

Synthesis of heterologous proteins in yeast is well known and described in the literature. Methods in Yeast Genetics, Sherman, F., et al., Cold Spring Harbor Laboratory, (1982) is a well-recognized work describing the various methods available to produce the polypeptide constructs in yeast. In yeast, vectors include Yeast Integrating plasmids (e.g., YIp5) and Yeast Replicating plasmids (the YRp series plasmids) and pGPD-2. Techniques for gene expression in various other microorganisms are described in, for example, Smith, Gene Expression in Recombinant Microorganisms (Bioprocess Technology, Vol. 22), Marcel Dekker, 1994. Examples of bacteria that are useful for expression include, but are not limited to, Escherichia, Enterobacter, Azotobacter, Erwinia, Bacillus, Pseudomonas, Klebsielia, Proteus, Salmonella, Serratia, Shigella, Rhizobia, Vitreoscilla, and Paracoccus. Filamentous fungi that are useful as expression hosts include, for example, Aspergillus, Trichoderma, Neurospora, Penicillium, Cephalosporium, Achlya, Podospora, Mucor, Cochliobolus, and Pyricularia. See, e.g., U.S. Pat. No. 5,679,543 and Stahl and Tudzynski, Eds., Molecular Biology in Filamentous Fungi, John Wiley & Sons, 1992.

Commonly used prokaryotic control sequences, e.g., promoters for transcription initiation, optionally with an operator, along with ribosome binding site sequences, include such commonly used promoters as the beta-lactamase (penicillinase) and lactose (lac) promoter systems (Change et al., Nature (1977) 198: 1056), the tryptophan (trp) promoter system (Goeddel et al., Nucleic Acids Res. (1980) 8: 4057), the tac promoter (DeBoer, et al., Proc. Natl. Acad. Sci. U.S.A. (1983) 80:21-25); and the lambda-derived PL promoter and N-gene ribosome binding site (Shimatake et al., Nature (1981) 292: 128). The particular promoter system is not critical; any available promoter that functions in prokaryotes and provides the desired level of activity can be used. Standard bacterial expression vectors include plasmids such as pBR322-based plasmids, e.g., pBLUESCRIPT™, pSKF, pET23D, lambda-phage derived vectors, and fusion expression systems such as GST and LacZ. Epitope tags can also be added to recombinant proteins to provide convenient methods of isolation, e.g., c-myc, HA-tag, 6-His tag (SEQ ID NO: 22), maltose binding protein, VSV-G tag, anti-DYKDDDDK tag (SEQ ID NO: 23), or any such tag, a large number of which are well known to those of skill in the art.

Either constitutive or regulated promoters can be used. Regulated promoters can be advantageous because the host cells can be grown to high densities before expression of the fusion polypeptides is induced. High level expression of heterologous proteins slows cell growth in some situations. An inducible promoter is a promoter that directs expression of a gene where the level of expression is alterable by environmental or developmental factors such as, for example, temperature, pH, anaerobic or aerobic conditions, light, transcription factors and chemicals. For E. coli and other bacterial host cells, inducible promoters are known to those of skill in the art. These include, for example, the lac promoter, the bacteriophage lambda PL promoter, the hybrid trp-lac promoter (Amann et al. (1983) Gene 25: 167; de Boer et al. (1983) Proc. Nat'l. Acad. Sci. USA 80: 21), and the bacteriophage T7 promoter (Studier et al. (1986) J. Mol. Biol.; Tabor et al. (1985) Proc. Nat'l Acad. Sci. USA 82: 1074-8). These promoters and their use are also discussed in Sambrook et al., supra.

Translational coupling may be used to enhance expression. The strategy uses a short upstream open reading frame derived from a highly expressed gene native to the translational system, which is placed downstream of the promoter, and a ribosome binding site followed after a few amino acid codons by a termination codon. Just prior to the termination codon is a second ribosome binding site, and following the termination codon is a start codon for the initiation of translation. The system dissolves secondary structure in the RNA, allowing for the efficient initiation of translation. See Squires, et. al. (1988), J. Biol. Chem. 263: 16297-16302.

The construction of securin-separase fusion proteins generally requires the use of vectors able to replicate in bacteria. Such vectors are commonly used in the art. Kits are commercially available for the purification of plasmids from bacteria (for example, EasyPrep™, FlexiPrep™, from Pharmacia Biotech; StrataClean™, from Stratagene; and, QIAexpress® Expression System, Qiagen). The isolated and purified plasmids can then be further manipulated to produce other plasmids, and used to transform cells.

The polypeptides described herein can be expressed intracellularly, or can be secreted from the cell. Intracellular expression often results in high yields. If necessary, the amount of soluble, active fusion polypeptide may be increased by performing refolding procedures (see, e.g., Sambrook et al., supra; Marston et al., Bio/Technology (1984) 2: 800; Schoner et al., Bio/Technology (1985) 3: 151

Once expressed, the polypeptides can be purified according to standard procedures of the art, including ammonium sulfate precipitation, affinity columns, column chromatography, gel electrophoresis and the like (see, generally, R. Scopes, Protein Purification, Springer-Verlag, N.Y. (1982), Deutscher, Methods in Enzymology Vol. 182: Guide to Protein Purification., Academic Press, Inc. N.Y. (1990)). Substantially pure compositions of at least about 90 to 95% homogeneity (e.g., 98 to 99% or higher homogeneity) are provided in certain embodiments. Once purified, partially or to homogeneity as desired, the polypeptides may then be used (e.g., in an inhibitor screen or mechanistic study).

To facilitate purification of the polypeptides, the nucleic acids that encode the polypeptides can also include a coding sequence for an epitope or “tag” for which an affinity binding reagent is available. Examples of suitable epitopes include the myc and V-5 reporter genes; expression vectors useful for recombinant production of fusion polypeptides having these epitopes are commercially available (e.g., Invitrogen (Carlsbad Calif.) vectors pcDNA3.1/Myc-His and pcDNA3.1/V5-His are suitable for expression in mammalian cells). Additional expression vectors suitable for attaching a tag to the fusion proteins of the invention, and corresponding detection systems are known to those of skill in the art, and several are commercially available (e.g., FLAG″ (Kodak, Rochester N.Y.)). Another example of a suitable tag is a polyhistidine sequence, which is capable of binding to metal chelate affinity ligands. Typically, six adjacent histidines (SEQ ID NO: 22) are used, although one can use more or less than six. Suitable metal chelate affinity ligands that can serve as the binding moiety for a polyhistidine tag include nitrilo-tri-acetic acid (NTA) (Hochuli, E. (1990) “Purification of recombinant proteins with metal chelating adsorbents” In Genetic Engineering: Principles and Methods, J. K. Setlow, Ed., Plenum Press, N.Y.; commercially available from Qiagen (Santa Clarita, Calif.)).

One of skill in the art would recognize that after biological expression or purification, the polypeptide constructs may possess a conformation substantially different than the native conformations of the constituent polypeptides. In this case, it may be necessary or desirable to denature and reduce the polypeptide and then to cause the polypeptide to re-fold into the preferred conformation. Methods of reducing and denaturing proteins and inducing re-folding are well known to those of skill in the art (See, Debinski et al. (1993) J. Biol. Chem. 268: 14065-14070; Kreitman and Pastan (1993) Bioconjug. Chem. 4: 581-585; and Buchner et al. (1992) Anal. Biochem. 205: 263-270). Debinski et al., for example, describe the denaturation and reduction of inclusion body proteins in guanidine-DTE. The protein is then refolded in a redox buffer containing oxidized glutathione and L-arginine.

In some embodiments, the methods for obtaining active separase include:
(a) co-expressing a separase and a securin, wherein the securin is linked to an unfoldase recognition site; and
(b) combining the co-expressed separase and securin with an unfoldase-peptidase complex;
thereby removing the securin and obtaining the active separase.

In some embodiments, the methods for obtaining active separase include:
(1) expressing a polypeptide comprising a securin fused to a separase; and
(2) removing the securin from the expressed polypeptide, thereby obtaining the active separase;
wherein the active separase is substantially free of the securin.

In some embodiments, the securin is fused to the separase via a linker comprising a protease recognition site, and removing the securin from the expressed polypeptide comprises cleaving the securin from the separase at the protease recognition site. In some embodiments, the polypeptide further comprises an unfoldase recognition site linked to the securin, and removing the securin from the expressed polypeptide comprises combining the expressed polypeptide with an unfoldase-peptidase complex. The methods of the present disclosure provide isolated active separase, which is substantially free of securin.

### IV. EXAMPLES

### Example. 1 Materials and Methods

Constructs, cloning and expression. Securin-separase fusion constructs were cloned into a pFastBac HT A vector with an L21 leader sequence added immediately upstream of the ORF³⁰. DNA encoding the N-terminal region of each protein (containing all or a subset of the following: LambdaO ClpX sequence, 2× StrepII tag, securin, Gly-Ser linker, TEV protease cleavage site, 3× FLAG tag) was codon optimized for insect cell expression and synthesized as a gBlocks gene fragment by Integrated DNA Technologies (IDT). Separase was amplified from a human cDNA library, and mutations were made using either gBlocks gene fragments or fragment amplification and then assembled using Gibson assembly. All constructs contained the S1126A mutation to prevent proline isomerization and subsequent aggregation¹³. Catalytically-dead separase constructs contained the C2029S mutation. For all constructs with an intact active site, the autocleavage sites were mutated by reversing the E and R residues for each of the three sites¹¹. All constructs were verified by full sequencing of the ˜7000 bp ORFs. The resulting plasmids were transformed into DH10Bac cells to generate bacmids through in vivo recombination. Purified bacmids were used to transfect Sf9 cells and generate P1 baculovirus. For protein expression, Sf9 cells were harvested 2-3 days after infection with P2 virus.

E. coli ClpX and ClpP-6His expression constructs were a generous gift from Andreas Martin. ClpX is the full-length, AKH version³¹, which we modified with a C-terminal 2× StrepII tag. TEV protease construct pRK793 was a gift from David Waugh (Addgene plasmid #8827; http://n2t.net/addgene:8827; RRID:Addgene_8827)³². TEV protease and ClpX were expressed in BL-21 DE3 E. coli at 30° C. for 4 h after induction with IPTG. ClpP was expressed in a BL21 ClpP knockout strain at 25° C. for 4 h after induction with IPTG.

The separase biosensor was generated as described by Shindo et al.²⁸. Specifically, Gibson cloning was performed to generate a final construct of pCMV-H2B-mRuby2-Scc1(142-467)-mNeonGreen in a plasmid backbone containing PGK-Neo. This was used as the template for all variations of the biosensor, which were also generated using Gibson cloning.

Protein purification. Securin-separase fusion protein and ClpX protein were purified on a StrepTrap column, with a lysis and wash buffer of 50 mM HEPES-KOH pH 7.8, 300 mM KCl, 0.1 mM EDTA-KOH, 0.5 mM TCEP, 10% glycerol. Proteins were eluted in one step in the same buffer containing 2.5 mM desthiobiotin. Securin-separase was used for ClpXP activation (see below) or buffer exchanged via PD-10 column into relevant buffers (see below), concentrated, frozen in aliquots of 100 μl or less in liquid nitrogen (LN₂), and stored at −80° C. Securin-separase used for negative-stain EM was additionally purified by size exclusion using a Superose 6 10/300 GL column pre-equilibrated in the following buffer: 25 mM HEPES pH 7.8, 75 mM KCl, 10 mM MgCl₂, 0.5 mM TCEP, 5% glycerol.

TEV protease and ClpP were purified on a HisTrap column. TEV protease buffers were 50 mM Tris-HCl pH 8, 200 mM NaCl, 10% glycerol, 0.5 mM TCEP, with 25 mM imidazole in the lysis and wash buffers and 800 mM imidazole in the elution buffer. ClpP buffers were 50 mM HEPES pH 7.8, 100 mM KCl, 400 mM NaCl, 10% glycerol, 0.5 mM TCEP, with 20 mM imidazole in the lysis and wash buffers and 500 mM imidazole in the elution buffer. TEV protease, ClpX and ClpP were each dialyzed overnight into 50 mM HEPES-KOH pH 7.5, 200 mM KCl, 25 mM MgCl₂, 0.1 mM EDTA, 0.5 mM TCEP, 10% glycerol. After dialysis, precipitate was pelleted by centrifugation and the supernatant frozen in aliquots of 250 μl or less in LN₂ and stored at −80° C.

Separase activation and purification. Securin-separase fusion was purified as described above. Eluted fractions were stored at 4° C. overnight, and then pooled and concentrated to ˜1 ml (˜2.5 mg/ml). The concentrated protein was incubated with 1 ml TEV protease (˜2.5 mg/ml) and 10 μl Benzonase added to 11.1 ml of 25 mM HEPES pH 7.8, 100 mM KCl, 10 mM MgCl₂, 10% glycerol for 1 h at 30° C. ClpX (1.7 ml, ˜1.6 mg/ml) and ClpP (830 μl, ˜2 mg/ml) were mixed and pre-incubated at 25° C. for over 30 min After the TEV protease incubation, 830 μl 100 mM ATP (in 25 mM HEPES pH 7.8, 100 mM KCl, 10 mM MgCl₂, 10% glycerol) was added to the securin-separase reaction mixture, followed by the pre-incubated ClpXP. After 1.5 h at 30° C., the mixture was filtered (0.2 μm) and run on a HisTrap column to remove ClpP and TEV protease. The flow-through was pooled, concentrated to less than 2.5 ml, and run over a PD-10 column to change the buffer to 50 mM HEPES-KOH pH 7.8, 300 mM KCl, 0.1 mM EDTA-KOH, 0.5 mM TCEP 10% glycerol. The protein was run on a StrepTrap column to remove ClpX and also any separase still bound by securin. The flow-through was pooled and concentrated to less than 1 ml, and loaded on a Superose 6 10/300 GL column pre-equilibrated in the following buffer: 25 mM HEPES pH 7.8, 75 mM KCl, 10 mM MgCl₂, 0.5 mM TCEP, 5% glycerol. The separase peak was pooled, concentrated, frozen in aliquots of 100 μl or less in LN₂ and stored at −80° C.

Electron Microscopy. Separase and the separase-securin complex were diluted to a nominal final concentration of 0.01 mg/ml in a buffer containing 25 mM HEPES-KOH pH 7.8, 75 mM KCl, 10 mM MgCl₂, 0.5 mM TCEP. For both samples, 3 μl were applied to carbon-coated 200-mesh copper grids (Ted Pella, Redding, Calif.) which had been glow discharged for 30 s. Specimens were stained as previously described³³ with a solution containing 2% (w/v) uranyl formate. Data were acquired with a Tecnai F20 Twin transmission electron microscope (FEI, Hillsboro, Oreg.) operating at 200 kV using SerialEM³⁴ and a nominal range of 0.9-1.9 μm under focus. Images were recorded on a TemCam-F816 CMOS camera (TVIPS, Gauting, Germany) at a nominal magnification of 50,000×, which corresponds to 1.57 Å/px at the detector level. For the separase sample, 337 images were collected (28,540 particles picked, ˜80 particles per image) and for the separase-securin complex 75 images were collected (26,077 particles picked, ˜350 particles per image) Immediately following image acquisition, micrographs were binned by two to give a final pixel size of 3.14 Å/px. The CTF was estimated using GCTF³⁵, and particles were picked using a reference free routine as implemented in Gautomatch (http://www.mrc-lmb.cam.ac.uk/kzhang/Gautomatch). Data were processed in a similar manner for each dataset, using Relion2³⁶ for 2D alignment and classification into 100 classes.

Analysis of DNA binding by fluorescence polarization. Double-stranded, 5′-fluorescein-labeled oligonucleotides were ordered from IDT. DNA was mixed with a dilution series of securin-separase C2029S with the following final conditions: 1 nM DNA in 25 mM HEPES pH 7.8, 50 mM KCl, 5 mM MgCl₂, 0.5 mM TCEP. Samples were incubated 30 min at 25° C. prior to measurement. Fluorescence polarization was measured on a Biotek Synergy H4 plate reader using excitation/emission of 485/528 nm at a gain of 70. Signal from wells with no protein were used to blank subtract the data, then the blank-subtracted fluorescence polarization was normalized relative to the average value at the highest protein concentration. Data were fit to a one-site binding model using GraphPad Prism.

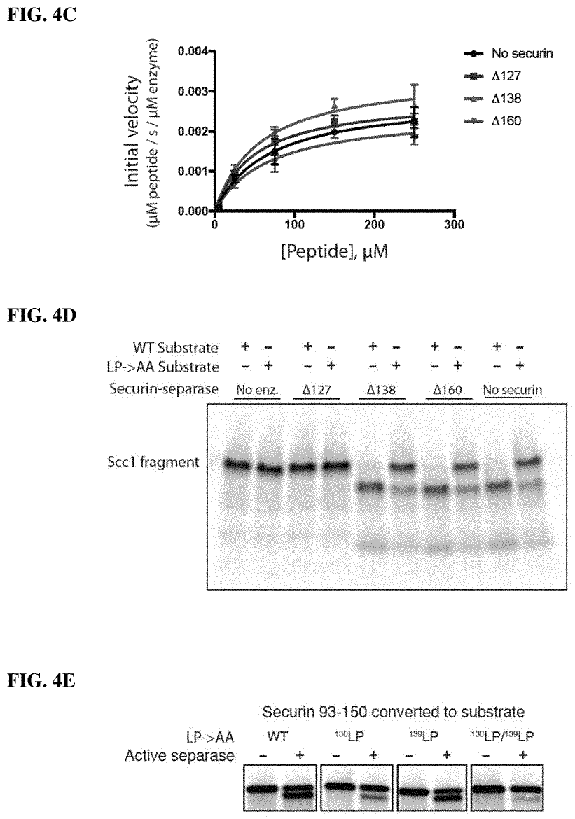

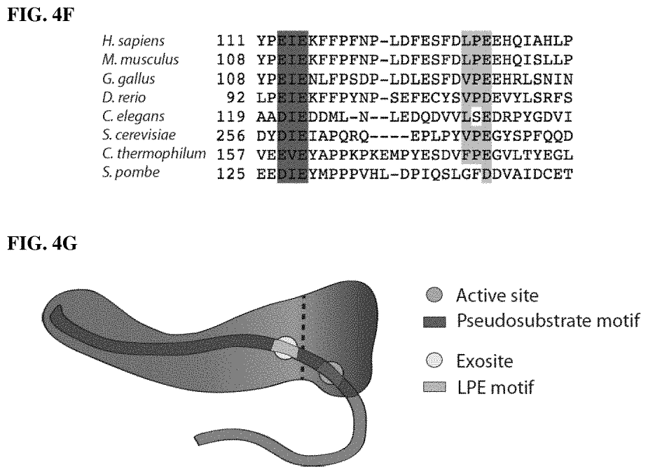

Scc1 cleavage assay. ³⁵S-methionine-labeled fragments of human Scc1 (and securin; FIG. 4E) were produced in rabbit reticulocyte lysates using the TnT Quick Coupled Transcription/Translation System (Promega). Variants were made by QuikChange mutagenesis or Gibson cloning. All variants included an N-terminal ZZ tag followed by a TEV protease cleavage site. Following translation in vitro, proteins were purified by immunoprecipitation on magnetic beads coated with anti-ZZ IgG, and eluted by TEV protease. Active separase (˜0.12 mg/ml) was mixed 1:1 with purified Scc1 substrate and incubated for 1 h at 25° C. Reaction products were analyzed by SDS-PAGE with BioRad 4-20% TXP gels and visualized with a Phosphorimager Gels were also stained with Coomassie Blue to confirm that enzyme concentration was the same in all reactions.

For experiments with securin-free separase, experiments were performed either with purified active separase or with activated separase but without downstream purification to remove TEV protease and ClpXP. The presence of ClpXP had no effect on the results. Additionally, in cases where ClpXP was present, apyrase was used to remove residual ATP and thereby prevent ClpXP activity.

Peptide cleavage assay. The following peptide, containing Scc1 site 1, was ordered from Genscript (>90% purity): Mca-DDREIMREGS-Dnp (SEQ ID NO: 25). Peptide was dissolved in DMSO at a concentration of 47.5 mM. The peptide was serially diluted into buffer (25 mM HEPES pH 7.8, 25 mM KCl, 0.5 mM TCEP) and mixed with active separase (either securin-free separase purified after TEV protease/ClpXP incubation or purified securinΔ-separase) at 0.1-0.5 mg/ml in the buffer: 25 mM HEPES pH 7.8, 75 mM KCl, 10 mM MgCl₂, 0.5 mM TCEP, 5% glycerol. The reaction was immediately monitored by fluorescence on a Biotek Synergy H4 plate reader, using an excitation of 328±20 nm and an emission filter of 393±20 nm (gain of 75). Fluorescence was monitored for 1 hour with 1 min reads. Data from 5-30 min was used for calculation of initial velocity.

To convert relative fluorescence units (RFU) to concentration of cleaved substrate, a standard curve was generated by incubating peptide with 0.1 mg/ml Trypsin for 2 h (to achieve full substrate cleavage) and then making a dilution series (in triplicate). Fluorescence was measured on the same day and at the same gain as in the kinetic assay. A plot of RFU vs concentration of cleaved peptide was fit with a linear regression and the slope taken as the conversion factor.

Separase concentrations were measured in triplicate on a Nanodrop spectrophotometer by absorbance at 280 nm, and evaluated using a theoretical extinction coefficient at A₂₈₀ (calculated according to the number of Trp and Tyr residues)³⁷. The data for the Michaelis-Menten curves were normalized by enzyme concentration. Data were fit to the Michaelis-Menten equation using GraphPad Prism. Error for reported k_{cat} incorporates the error in protein concentration.

Biosensor expression and microscopy. Second-generation lentiviruses were generated by transient co-transfection of 293T cells in DMEM+10% FBS, using a three-plasmid combination: one well in a 6-well dish containing 1×10⁶ 293 T cells was transfected using PEI with 0.5 μg lentiviral vector, 0.5 μg psPAX and 0.5 μg pMD2.G. Supernatants were collected every 24 h between 24 and 72 h after transfection and frozen at −80° C.

For biosensor expression, U2OS cells growing in McCoy's media+10% FBS were plated in a 6-well dish at 1×10⁶ cells per well. The following day, 0.5 ml lentivirus was added. After 48 h incubation, media was removed and cells were washed with PBS. Next, fresh media with 500 μg/ml Geneticin was added to the cells to select for transduced cells. After 1-2 weeks of selection, cell lines were expanded for FACS analysis: cells were re-suspended in FACS sorting buffer (PBS [Ca²⁺/Mg²⁺-free], 1 mM EDTA, 25 mM HEPES, 1% FBS) and filtered through a 50 μM filter. These cells were then sorted on a Sony SH800 Cell Sorter, selecting for cells with moderate levels of expression.

For microscopy, U2OS cells stably expressing the biosensor were plated in 24-well glass-bottom dishes (Mattek P24G-1.0-10-F) and allowed to adhere overnight. Media was removed and the cells were washed with PBS. Media was then replaced with Opti-Mem supplemented with 10% FBS. Cells were imaged at 37° C. with 5% CO₂ on a Nikon Ti inverted microscope equipped with CSU-22 spinning disk confocal and EMCCD camera. Mitotic cells were identified and time points were taken every 2.5 min. For data analysis, images were processed using ImageJ software as follows. Metaphase cells were identified by visual inspection of DNA labeled with H2B-mRuby2. The mean fluorescence intensities of GFP and RFP associated with DNA was then determined and the ratio of GFP to RFP was calculated. The ratio of fluorescent intensities was normalized to metaphase ratios, as it was assumed that the biosensor was intact at this stage. For each post-metaphase time point, the GFP:RFP ratio was determined for the brightest set of chromosomes and normalized against the GFP:RFP metaphase timepoint.

### Example 2. A Novel Strategy to Produce Active Human Separase for Studies In Vitro

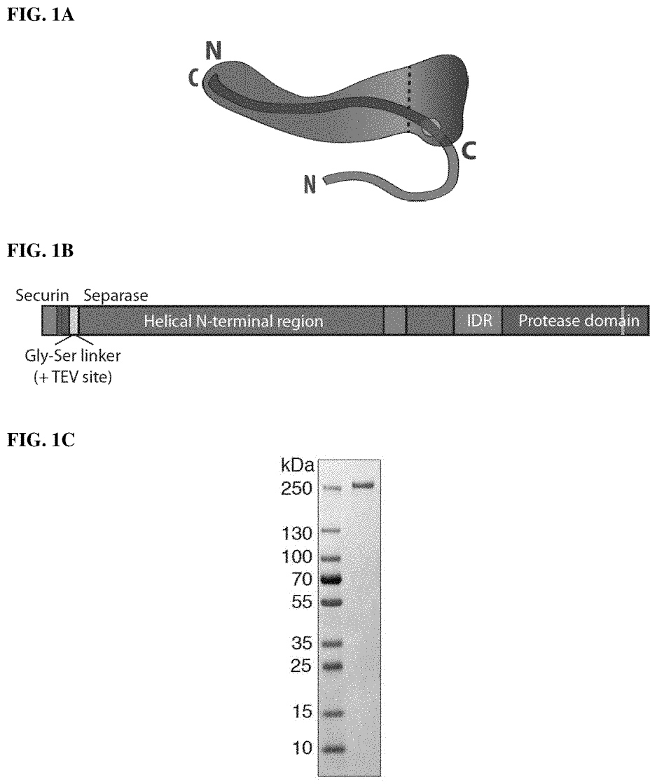

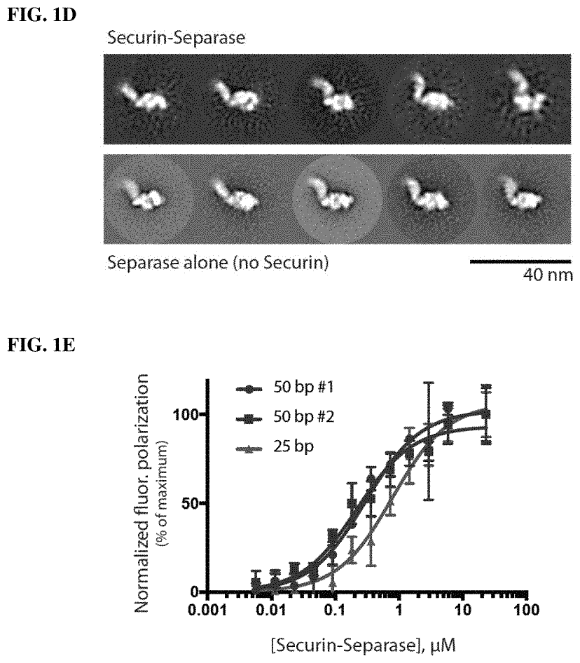

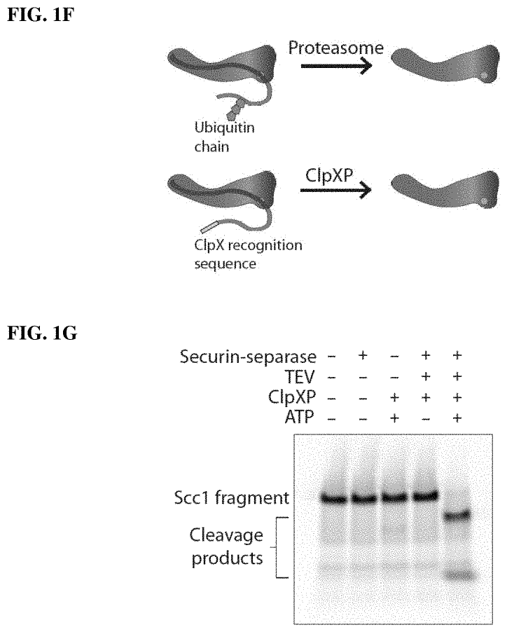

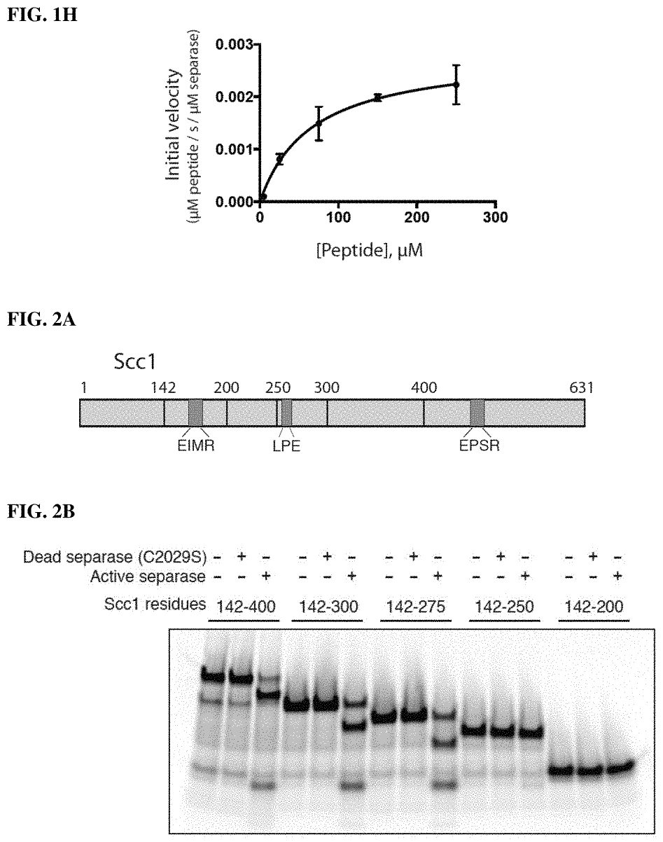

Production of active human separase protein at a purity and scale sufficient for biophysical characterization was sought, and expression in Sf9 insect cells with recombinant baculoviruses²¹ was employed. First, a gene fusion between the securin C-terminus and the separase N-terminus, separated by a Gly-Ser linker (FIG. 1B) was created. There is evidence that securin is a folding chaperone of separase^{17,22} and that these protein termini are co-localized²¹. Expression of the fusion construct led to protein levels that were significantly higher than those seen when securin and separase were co-expressed in Sf9 cells. Yield was improved further by N-terminal truncation of securin to remove its APC/C degrons and by elimination of the separase autocleavage sites by mutation.

Purified securin-separase (FIG. 1C) was characterized by negative-stain electron microscopy (EM) (FIG. 1D, top). The sample was monodisperse, and class averages were consistent with existing EM data for human securin-separase^{6,21}.

Human securin-separase has been demonstrated to bind DNA in a non-sequence specific manner¹⁵. The fusion securin-separase complex was evaluated for similar behavior. Binding of securin-separase to a fluorescently-labeled 50 base-pair double-stranded DNA molecule was evaluated by monitoring fluorescence polarization as a function of protein concentration (FIG. 1E). The data fit well to a one site specific-binding model with a K_{D} of 300 nM+/−100 nM. A DNA molecule with the same base composition but different sequence yielded a similar K_{D} (220 nM+/−60 nM). Because the separase-DNA interaction is not sequence-specific, it was expected that the measured affinity would depend on length, with shorter DNA molecules exhibiting lower affinities. Indeed, a 25 base-pair DNA molecule bound with a lower affinity (K_{D}=800 nM+/−300 nM).

Next, a method for activating separase using purified components was developed, rather than the traditional method of using the APC/C-proteasome system in Xenopus egg extract. Analogous to the proteasome, the ClpXP protein complex consists of an unfoldase (the ATPase ClpX) and a peptidase (ClpP)²³. However, whereas the proteasome interacts with ubiquitin to determine its targets, ClpXP engages with specific short amino acid sequences²³ (FIG. 1F). Additionally, E. coli ClpXP can be produced recombinantly much more readily than the proteasome. There is also precedent for the use of ClpXP to selectively remove a protein from a protein complex²⁴. A ClpXP recognition site was added at the N-terminus of securin in the fusion construct, as well as a TEV protease cleavage site in the linker between securin and separase. Following purification and cleavage with TEV protease, incubation with purified ClpXP removed securin and activated separase, as evaluated by cleavage of an Scc1 fragment in vitro (FIG. 1G). Separase also cleaved a catalytically-dead separase with intact autocleavage sites, demonstrating that separase autocleavage can occur in trans.

The ClpXP-activated separase was re-purified to remove TEV protease, ClpXP, and any separase still bound by securin. This purification yielded sufficient active separase to measure protein concentration spectroscopically and to perform basic biophysical characterization. First, Michaelis-Menten analysis was used to analyze the kinetics of the interaction between the enzyme active site and a cleavage substrate. These experiments were performed with a substrate peptide encompassing the best-characterized separase cleavage site in human Scc1 (¹⁶⁹EIMR (SEQ ID NO: 27), or “site 1”) flanked by a FRET dye-quencher pair (FIG. 1H). The results fit well to a standard Michaelis-Menten curve, yielding a KM of 70±30 μM and a kcal of 3×10⁻³±1×10⁻³ sec⁻¹ (or 10±3 hour⁻¹). These results are consistent with a previous analysis of active separase reaction kinetics²⁵.

Finally, the apo separase was evaluated using negative-stain EM (FIG. 1D, bottom). The sample was monodisperse and indistinguishable from the securin-separase complex at this resolution, indicating that separase does not undergo a large-scale conformational change upon securin removal.

### Example 3. Scc1 Residues Distant from the Separase Cleavage Site are Critical for Cleavage In Vitro

These studies revealed that separase activity toward a minimal cleavage site exhibits a very low catalytic rate²⁶, suggesting that cleavage rate is somehow enhanced in the cell. Though it is possible that DNA binding (FIG. 1E) provides the extra affinity needed to boost function in vivo, this would be highly nonspecific if this were the only mechanism. The possibility that separase has a more specific substrate docking site was therefore explored.

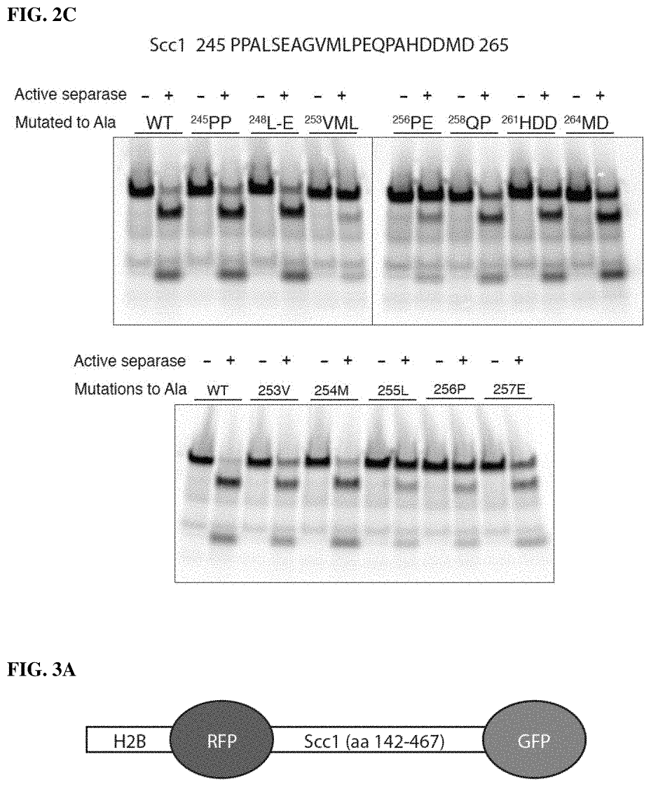

The two separase cleavage sites in Scc1 are located within a large region of predicted disorder between the terminal regions that interact with the Smc3 and Smc1 subunits of cohesin²⁷. To investigate whether local sequence context accelerates the cleavage of Scc1, a series of Scc1 truncations was evaluated with an in vitro cleavage assay (FIG. 2A). The starting point for this assay was an internal Scc1 fragment (amino acids 142-400), which was chosen after more robust cleavage of Scc1 by separase was observed when the terminal regions that interact with Smc3 and Smc1 were removed. This internal fragment does not contain site 2 (⁴⁴⁷EPSR (SEQ ID NO: 28)), and so it is cleavage at site 1 (¹⁶⁹EIMR (SEQ ID NO: 27)) that was evaluated here. However, even when site 2 is present, it did not appear to be cleaved in this assay, perhaps because cleavage at site 2 requires other factors, such as adjacent phosphorylation by Plk1¹⁰.

An abrupt reduction in cleavage of the Scc1 fragment upon C-terminal truncation from residues 275 to 250 (FIG. 2B) was observed, suggesting the presence of a separase-binding motif in this region of Scc1. Note that this assay has lower sensitivity than the above peptide cleavage assay, explaining why cleavage of the smallest fragments is not observed even though they contain the peptide sequence. Alanine scanning revealed that the most critical residues for enhanced activity were a Leu-Pro sequence at residues 255 and 256, with a contribution from Glu 257 (FIG. 2C). These residues are referred to hereinafter as the LPE motif.

### Example 4. The LPE Motif in Scc1 is Important for Cleavage by Separase In Vivo

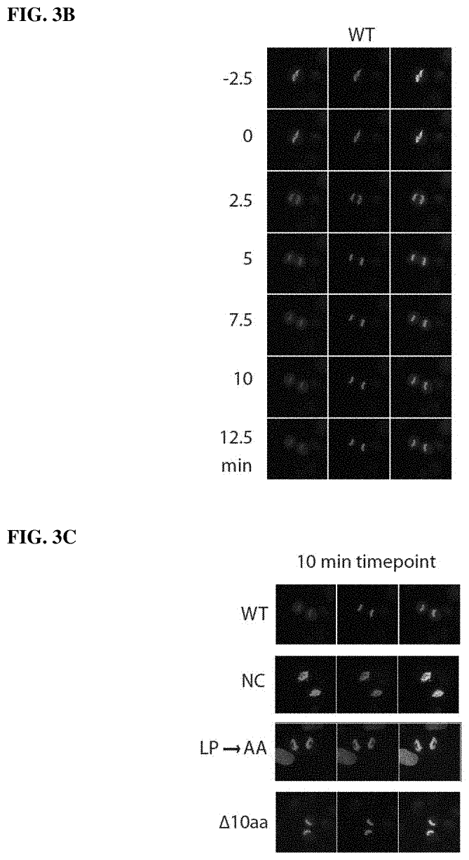

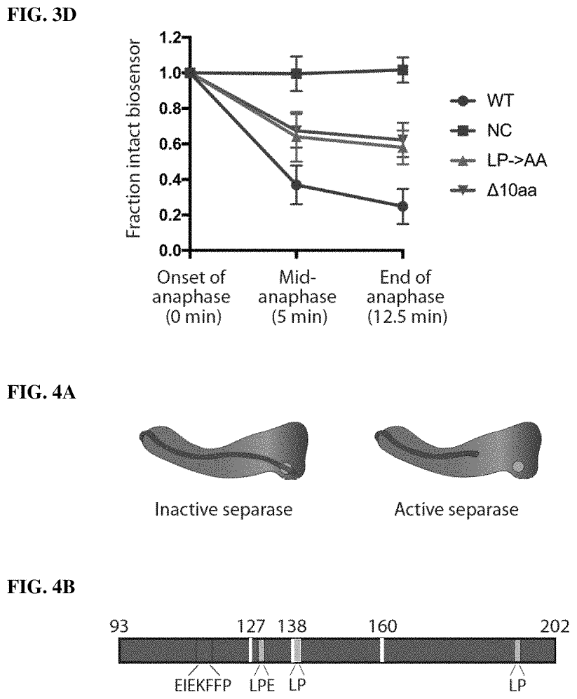

Having demonstrated the importance of the LPE motif for separase cleavage of Scc1 in vitro, its importance in vivo was tested. A previously described separase biosensor in human U2OS cells (FIG. 3A)²⁸ was recreated for this purpose. With wild-type Scc1 (aa 142-467), efficient biosensor cleavage was observed during anaphase (FIG. 3B). Strikingly, the double point mutation²⁵⁵Leu-Pro 4 Ala-Ala reduced cleavage efficiency by 50% (FIGS. 3C, D). A biosensor containing a 10 amino acid deletion centered on ²⁵⁵Leu-Pro yielded identical results as the double alanine mutation, confirming that these two residues are key requirements for this interaction (FIGS. 3C, D). Although separase cleavage site 2 is present in this biosensor, it was not cleaved in the assay, nor was it cleaved in a longer version of the biosensor that extended 123 amino acids beyond site 2. Therefore, the observation that ²⁵⁵Leu-Pro promotes biosensor cleavage is specific to site 1.

### Example 5. Securin Interferes with Separase Binding to the LPE Motif in Scc1

The results above suggest that an exosite on separase interacts with the LPE motif in Scc1, resulting in higher substrate affinity and more efficient cleavage. An intriguing possibility is that securin binding prevents this interaction, providing an additional mechanism by which securin inhibits Scc1 cleavage. To address this possibility, securin-separase fusion proteins were created in which securin was truncated after the pseudosubstrate sequence that binds the separase active site (FIG. 4A). The likely pseudosubstrate sequence (¹¹³EIEKFFP (SEQ ID NO: 26)) was identified previously based on homology between human and C. thermophilum securins⁴. It was hypothesized that removal of the pseudosubstrate motif would relieve the inhibition caused by securin directly blocking the separase active site, but that it would retain any effect of securin binding elsewhere on separase. It was also hypothesized that the fusion approach would have the benefit of generating an extremely high local concentration of securin, compared to adding securin in trans. Three securin truncations were tested, having securin residues 127-202, 138-202 or 160-202 covalently linked to separase via a flexible glycine-serine linker. The constructs are referred to hereinafter as securinΔ127-separase, securinΔ138-separase, and securinΔ160-separase, respectively (FIG. 4B).

It was first asked whether removal of the securin pseudosubstrate region from the active site was sufficient to yield a cleavage-competent active site. Michaelis-Menten analyses with the peptide assay described above (FIG. 1G) showed that there were no significant differences between the peptide cleavage activities of the three securinΔ-separase proteins and separase with no securin bound (FIG. 4C). These results confirmed that the pseudosubstrate sequence blocks the active site, and they also suggested that securin binding outside the active site does not impair catalysis through some allosteric mechanism.

Experiments were then conducted to test whether the securinΔ-separase constructs were able to cleave the Scc1 fragment in the gel-based assay, and whether this cleavage was sensitive to mutation of the LPE motif (FIG. 4D). SecurinΔ138-separase and securinΔ160-separase exhibited efficient cleavage of the Scc1 substrate, and in both cases activity was reduced by mutation of ²⁵⁵Leu-Pro. However, securinΔ127-separase exhibited no cleavage activity in this assay. This result strongly suggests that securin interferes with separase binding to the LPE motif on Scc1, and that this interference is localized to a region of securin between residues 127 and 138. Intriguingly, this region of securin contains an LPE motif (residues 130-132).

An approach for investigating the importance of ¹³⁰LPE for securin binding to separase was developed. It is known that fungal securin can be converted to a separase substrate by making mutations that convert the pseudosubstrate site into a cleavage site⁴. The equivalent mutations were made in human securin (¹¹⁸FP to RE) and this securin^{RE} mutant was used to test the importance of ¹³⁰LPE for securin engagement with separase. An LP sequence a few residues further downstream (¹³⁹LP) was also tested. In initial experiments, a securin^{RE} fragment containing residues 93-202 was cleaved efficiently by separase, but mutation of either LP sequence had no effect, presumably because this fragment of securin makes too many contacts with separase for individual point mutations to significantly weaken affinity. A securin^{RE} fragment containing residues 93-150 was then tested. This fragment was 50% cleaved by separase, and mutation of ¹³⁰LP significantly impaired cleavage (FIG. 4E). Mutation of ¹³⁹LP had no effect, except when combined with mutation of ¹³⁰LP. These results indicate that the ¹³⁰LPE motif of securin interacts with separase.

Consistent with its importance in the regulation of separase, the LPE sequence immediately downstream of the pseudosubstrate motif is conserved in securin from vertebrates and in some lower eukaryotes (FIG. 4F). Budding yeast securin carries a VPE sequence at this location, and the crystal structure of the yeast separase-securin complex indicates that the valine and proline interact with the surface of separase adjacent to the catalytic domains. This region of the human separase differs from the yeast separase, precluding straightforward prediction of the precise LPE motif-binding site in the human protein.

The major current method for separase productions depends on removal of securin from small amounts of securin-separase complex using extracts of frog eggs. This generates very small amounts of impure enzyme and is not widely used. There are no currently-available commercial sources of separase, and no previous method could be scaled up to produce large amounts of homogeneous enzyme. As such, the biochemical behavior of the enzyme has remained largely unexplored in the 20 years since it was discovered. The separase constructs of the present disclosure allow for the new discovery of important enzyme characteristics such as the LPE motif described herein.

### V. References

1. Uhlmann, et al. Nature 400, 37-42, doi:10.1038/21831 (1999).
2. Uhlmann, et al. Cell 103, 375-386 (2000).
3. Nasmyth & Haering. Annu Rev Genet 43, 525-558, doi:10.1146/annurev-genet-102108-134233 (2009).
4. Lin et al. Nature 532, 131-134, doi:10.1038/nature17402 (2016).
5. Luo & Tong. Nature 542, 255-259, doi:10.1038/nature21061 (2017).
6. Boland, et al. Nature Structural & Molecular Biology 24, 414-418, doi:10.1038/nsmb.3386 (2017).
7. Luo & Tong. Curr Opin Struct Biol 49, 114-122, doi:10.1016/j.sbi.2018.01.012 (2018).
8. Hauf, et al. Science 293, 1320-1323, doi:10.1126/science.1061376 (2001).
9. Alexandru, et al. Cell 105, 459-472, doi:10.1016/S0092-8674(01)00362-2 (2001).
10. Hauf, et al. PLoS biology 3, e69, doi:10.1371/journal.pbio.0030069 (2005).
11. Waizenegger, et al. Current biology 12, 1368-1378 (2002).
12. Csizmok, et al. Journal of the American Chemical Society 130, 16873-16879, doi:10.1021/ja805510b (2008).
13. Hellmuth, et al. Molecular Cell 58, 1-12, doi:10.1016/j.molcel.2015.03.025 (2015).
14. Jabaiah, et al. Biological Chemistry 393, 661-616, doi:10.1515/hsz-2012-0162 (2012).
15. Sun, et al. Cell 137, 123-132, doi:10.1016/j.cell.2009.01.040 (2009).
16. Ciosk, et al. Cell 93, 1067-1076, doi:10.1016/50092-8674(00)81211-8 (1998).
17. Hellmuth, et al. Journal of Biological Chemistry 290, 8002-8010, doi:10.1074/jbc.M114.615310 (2015).
18. Stemmann, et al. Cell 107, 715-726 (2001).
19. Gorr, et al. Molecular Cell 19, 135-141, doi:10.1016/j.molcel.2005.05.022 (2005).
20. Hellmuth, et al. The EMBO Journal 33, 1134-1147, doi:10.1002/embj.201488098 (2014).
21. Viadiu, et al. Nature Structural & Molecular Biology 12, 552-553, doi:10.1038/nsmb935 (2005).
22. Pfleghaar, et al. PLoS biology 3, e416, doi:10.1371/journal.pbio.0030416 (2005).
23. Baker & Sauer. BBA—Molecular Cell Research 1823, 15-28, doi:10.1016/j.bbamcr.2011.06.007 (2012).
24. Moore, et al. Proc Natl Acad Sci USA 105, 11685-11690, doi:10.1073/pnas.0805633105 (2008).
25. Zhang, et al. Journal of Biomolecular Screening 19, 878-889, doi:10.1177/1087057114520972 (2014).
26. Bar-Even, et al. Biochemistry 50, 4402-4410, doi:10.1021/bi2002289 (2011).
27. Uhlmann. Nature Reviews Molecular Cell Biology 17, 399-412, doi:10.1038/nrm.2016.30 (2016).
28. Shindo, et al. Developmental Cell 23, 112-123, doi:10.1016/j.devce1.2012.06.015 (2012).
29. Kudo, et al. J Cell Sci 122, 2686-2698, doi:10.1242/jcs.035287 (2009).
30. Sano, et al. FEBS letters 532, 143-146 (2002).
31. Farrell, et al. Molecular Cell 25, 161-166, doi:10.1016/j.molcel.2006.11.018 (2007).
32. Kapust, et al. Protein Eng 14, 993-1000, doi:10.1093/protein/14.12.993 (2001).
33. Ohi, et al. Biological procedures online 6, 23-34, doi:10.1251/bpo70 (2004).
34. Mastronarde. J Struct Biol 152, 36-51, doi:10.1016/j.jsb.2005.07.007 (2005).
35. Zhang. J Struct Biol 193, 1-12, doi:10.1016/j.jsb.2015.11.003 (2016).
36. Scheres. J Struct Biol 180, 519-530, doi:10.1016/j.jsb.2012.09.006 (2012).
37. Edelhoch. Biochemistry 6, 1948-1954 (1967).

Although the foregoing has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

VI. INFORMAL SEQUENCE LISTING
Human (Homo sapiens) separase
SEQ ID NO: 1
MRSFKRVNFGTLLSSQKEAEELLPDLKEFLSNPPA
GFPSSRSDAERRQACDAILRACNQQLTAKLACPRH
LGSLLELAELACDGYLVSTPQRPPLYLERILFVLL
RNAAAQGSPEATLRLAQPLHACLVQCSREAAPQDY
EAVARGSFSLLWKGAEALLERRAAFAARLKALSFL
VLLEDESTPCEVPHFASPTACRAVAAHQLFDASGH
GLNEADADFLDDLLSRHVIRALVGERGSSSGLLSP
QRALCLLELTLEHCRRFCWSRHHDKAISAVEKAHS
YLRNTNLAPSLQLCQLGVKLLQVGEEGPQAVAKLL
IKASAVLSKSMEAPSPPLRALYESCQFFLSGLERG
TKRRYRLDAILSLFAFLGGYCSLLQQLRDDGVYGG
SSKQQQSFLQMYFQGLHLYTVVVYDFAQGCQIVDL
ADLTQLVDSCKSTVVWMLEALEGLSGQELTDHMGM
TASYTSNLAYSFYSHKLYAEACAISEPLCQHLGLV
KPGTYPEVPPEKLHRCFRLQVESLKKLGKQAQGCK
MVILWLAALQPCSPEHMAEPVTFWVRVKMDAARAG
DKELQLKTLRDSLSGWDPETLALLLREELQAYKAV
RADTGQERFNIICDLLELSPEETPAGAWARATHLV
ELAQVLCYHDFTQQTNCSALDAIREALQLLDSVRP
EAQARDQLLDDKAQALLWLYICTLEAKIQEGIERD
RRAQAPGNLEEFEVNDLNYEDKLQEDRFLYSNIAF
NLAADAAQSKCLDQALALWKELLTKGQAPAVRCLQ
QTAASLQILAALYQLVAKPMQALEVLLLLRIVSER
LKDHSKAAGSSCHITQLLLTLGCPSYAQLHLEEAA
SSLKHLDQTTDTYLLLSLTCDLLRSQLYWTHQKVT
KGVSLLLSVLRDPALQKSSKAWYLLRVQVLQLVAA
YLSLPSNNLSHSLWEQLCAQGWQTPEIALIDSHKL
LRSIILLLMGSDILSTQKAAVETSFLDYGENLVQK
WQVLSEVLSCSEKLVCHLGRLGSVSEAKAFCLEAL
KLTTKLQIPRQCALFLVLKGELELARNDIDLCQSD
LQQVLFLLESCTEFGGVTQHLDSVKKVHLQKGKQQ
AQVPCPPQLPEEELFLRGPALELVATVAKEPGPIA
PSTNSAPVLKTKPQPIPNFLSHSPTCDCSLCASPV
LTAVCLRWVLVTAGVRLAMGHQAQGLDLLQVVLKG
CPEAAERLTQALQASLNHKTPPSLVPSLLDEILAQ
AYTLLALEGLNQPSNESLQKVLQSGLKFVAARIPH
LEPWRASLLLIWALTKLGGLSCCTTQLFASSWGWQ
PPLIKSVPGSEPSKTQGQKRSGRGRQKLASAPLSL
NNTSQKGLEGRGLPCTPKPPDRIRQAGPHVPFTVF
EEVCPTESKPEVPQAPRVQQRVQTRLKVNFSDDSD
LEDPVSAEAWLAEEPKRRGTASRGRGRARKGLSLK
TDAVVAPGSAPGNPGLNGRSRRAKKVASRHCEERR
PQRASDQARPGPRIMETIPEEELTDNWRKMSFRIL
EGSDGEDSASGGKTPAPGPEAASGEWRLLELDSSK
KKLPSPCPDKESDKDLGPRLQLPSAPVATGLSTLD
SICDSLSVAFRGISHCPPSGLYAHLCRFLALCLGH
RDPYATAFLVTESVSITCRHQLLTHLHRQLSKAQK
HRGSLEIADQLQGLSLQEMPGDVPLARIQRLFSFR
ALESGHFPQPEKESFQERLALIPSGVTVCVLALAT
LQPGTVGNTLLLTRLEKDSPPVSVQIPTGQNKLHL
RSVLNEFDAIQKAQKENSSCTDKREWWTGRLALDH
RMEVLIASLEKSVLGCWKGLLLPSSEEPGPAQEAS
RLQELLQDCGWKYPDRTLLKIMLSGAGALTPQDIQ
ALAYGLCPTQPERAQELLNEAVGRLQGLTVPSNSH
LVLVLDKDLQKLPWESMPSLQALPVTRLPSFRFLL
SYSIIKEYGASPVLSQGVDPRSTFYVLNPHNNLSS
TEEQFRANFSSEAGWRGVVGEVPRPEQVQEALTKH
DLYIYAGHGAGARFLDGQAVLRLSCRAVALLFGCS
SAALAVHGNLEGAGIVLKYIMAGCPLFLGNLWDVT
DRDIDRYTEALLQGWLGAGPGAPLLYYVNQARQAP
RLKYLIGAAPIAYGLPVSLR
Human (Homo sapiens) securin
SEQ ID NO: 2
MATLIYVDKENGEPGTRVVAKDGLKLGSGPSIKAL
DGRSQVSTPRFGKTFDAPPALPKATRKALGTVNRA
TEKSVKTKGPLKQKQPSFSAKKMTEKTVKAKSSVP
ASDDAYPEIEKFFPFNPLDFESFDLPEEHQIAHLP
LSGVPLMILDEERELEKLFQLGPPSPVKMPSPPWE
SNLLQSPSSILSTLDVELPPVCCDIDI
Securin-separase
SEQ ID NO: 3
MTNTAKILNFGRWSHPQFEKGSAGSAAGSGAGWSH
PQFEKGSASMTEKTVKAKSSVPASDDAYPEIEKFF
PFNPLDFESFDLPEEHQIAHLPLSGVPLMILDEER
ELEKLFQLGPPSPVKMPSPPWESNLLQSPSSILST
LDVELPPVCCDIDIGGSGGSGGGSGGGSGENLYFQ
GDYKDHDGDYKDHDIDYKDDDDKSGPMRSFKRVNF
GTLLSSQKEAEELLPDLKEFLSNPPAGFPSSRSDA
ERRQACDAILRACNQQLTAKLACPRHLGSLLELAE
LACDGYLVSTPQRPPLYLERILFVLLRNAAAQGSP
EATLRLAQPLHACLVQCSREAAPQDYEAVARGSFS
LLWKGAEALLERRAAFAARLKALSFLVLLEDESTP
CEVPHFASPTACRAVAAHQLFDASGHGLNEADADF
LDDLLSRHVIRALVGERGSSSGLLSPQRALCLLEL
TLEHCRRFCWSRHHDKAISAVEKAHSYLRNTNLAP
SLQLCQLGVKLLQVGEEGPQAVAKLLIKASAVLSK
SMEAPSPPLRALYESCQFFLSGLERGTKRRYRLDA
ILSLFAFLGGYCSLLQQLRDDGVYGGSSKQQQSFL
QMYFQGLHLYTVVVYDFAQGCQIVDLADLTQLVDS
CKSTVVWMLEALEGLSGQELTDHMGMTASYTSNLA
YSFYSHKLYAEACAISEPLCQHLGLVKPGTYPEVP
PEKLHRCFRLQVESLKKLGKQAQGCKMVILWLAAL
QPCSPEHMAEPVTFWVRVKMDAARAGDKELQLKTL
RDSLSGWDPETLALLLREELQAYKAVRADTGQERF
NIICDLLELSPEETPAGAWARATHLVELAQVLCYH
DFTQQTNCSALDAIREALQLLDSVRPEAQARDQLL
DDKAQALLWLYICTLEAKIQEGIERDRRAQAPGNL
EEFEVNDLNYEDKLQEDRFLYSNIAFNLAADAAQS
KCLDQALALWKELLTKGQAPAVRCLQQTAASLQIL
AALYQLVAKPMQALEVLLLLRIVSERLKDHSKAAG
SSCHITQLLLTLGCPSYAQLHLEEAASSLKHLDQT
TDTYLLLSLTCDLLRSQLYWTHQKVTKGVSLLLSV
LRDPALQKSSKAWYLLRVQVLQLVAAYLSLPSNNL
SHSLWEQLCAQGWQTPEIALIDSHKLLRSIILLLM
GSDILSTQKAAVETSFLDYGENLVQKWQVLSEVLS
CSEKLVCHLGRLGSVSEAKAFCLEALKLTTKLQIP
RQCALFLVLKGELELARNDIDLCQSDLQQVLFLLE
SCTEFGGVTQHLDSVKKVHLQKGKQQAQVPCPPQL
PEEELFLRGPALELVATVAKEPGPIAPSTNSAPVL
KTKPQPIPNFLSHSPTCDCSLCASPVLTAVCLRWV
LVTAGVRLAMGHQAQGLDLLQVVLKGCPEAAERLT
QALQASLNHKTPPSLVPSLLDEILAQAYTLLALEG
LNQPSNESLQKVLQSGLKFVAARIPHLEPWRASLL
LIWALTKLGGLSCCTTQLFASSWGWQPPLIKSVPG
SEPSKTQGQKRSGRGRQKLASAPLSLNNTSQKGLE
GRGLPCTPKPPDRIRQAGPHVPFTVFEEVCPTESK
PEVPQAPRVQQRVQTRLKVNFSDDSDLEDPVSAEA
WLAEEPKRRGTASRGRGRARKGLSLKTDAVVAPGS
APGNPGLNGRSRRAKKVASRHCEERRPQRASDQAR
PGPRIMETIPEEELTDNWRKMSFRILEGSDGEDSA
SGGKTPAPGPEAASGEWRLLELDSSKKKLPSPCPD
KESDKDLGPRLQLPSAPVATGLSTLDSICDSLSVA
FRGISHCPPSGLYAHLCRFLALCLGHRDPYATAFL
VTESVSITCRHQLLTHLHRQLSKAQKHRGSLEIAD
QLQGLSLQEMPGDVPLARIQRLFSFRALESGHFPQ
PEKESFQERLALIPSGVTVCVLALATLQPGTVGNT
LLLTRLEKDSPPVSVQIPTGQNKLHLRSVLNEFDA
IQKAQKENSSCTDKREWWTGRLALDHRMEVLIASL
EKSVLGCWKGLLLPSSEEPGPAQEASRLQELLQDC
GWKYPDRTLLKIMLSGAGALTPQDIQALAYGLCPT
QPERAQELLNEAVGRLQGLTVPSNSHLVLVLDKDL
QKLPWESMPSLQALPVTRLPSFRFLLSYSIIKEYG
ASPVLSQGVDPRSTFYVLNPHNNLSSTEEQFRANF
SSEAGWRGVVGEVPRPEQVQEALTKHDLYIYAGHG
AGARFLDGQAVLRLSCRAVALLFGCSSAALAVHGN
LEGAGIVLKYIMAGCPLFLGNLWDVTDRDIDRYTE
ALLQGWLGAGPGAPLLYYVNQARQAPRLKYLIGAA
PIAYGLPVSLR
SecurinΔ127-separase
SEQ ID NO: 4
MHWSHPQFEKGSAGSAAGSGAGWSHPQFEKGSSTA
SENLYFQGSFDLPEEHQIAHLPLSGVPLMILDEER
ELEKLFQLGPPSPVKMPSPPWESNLLQSPSSILST
LDVELPPVCCDIDIGGSGGSGGGSGGGSGGGSGGD
YKDHDGDYKDHDIDYKDDDDKSGPMRSFKRVNFGT
LLSSQKEAEELLPDLKEFLSNPPAGFPSSRSDAER
RQACDAILRACNQQLTAKLACPRHLGSLLELAELA
CDGYLVSTPQRPPLYLERILFVLLRNAAAQGSPEA
TLRLAQPLHACLVQCSREAAPQDYEAVARGSFSLL
WKGAEALLERRAAFAARLKALSFLVLLEDESTPCE
VPHFASPTACRAVAAHQLFDASGHGLNEADADFLD
DLLSRHVIRALVGERGSSSGLLSPQRALCLLELTL
EHCRRFCWSRHHDKAISAVEKAHSYLRNTNLAPSL
QLCQLGVKLLQVGEEGPQAVAKLLIKASAVLSKSM
EAPSPPLRALYESCQFFLSGLERGTKRRYRLDAIL
SLFAFLGGYCSLLQQLRDDGVYGGSSKQQQSFLQM
YFQGLHLYTVVVYDFAQGCQIVDLADLTQLVDSCK
STVVWMLEALEGLSGQELTDHMGMTASYTSNLAYS
FYSHKLYAEACAISEPLCQHLGLVKPGTYPEVPPE
KLHRCFRLQVESLKKLGKQAQGCKMVILWLAALQP
CSPEHMAEPVTFWVRVKMDAARAGDKELQLKTLRD
SLSGWDPETLALLLREELQAYKAVRADTGQERFNI
ICDLLELSPEETPAGAWARATHLVELAQVLCYHDF
TQQTNCSALDAIREALQLLDSVRPEAQARDQLLDD
KAQALLWLYICTLEAKIQEGIERDRRAQAPGNLEE
FEVNDLNYEDKLQEDRFLYSNIAFNLAADAAQSKC
LDQALALWKELLTKGQAPAVRCLQQTAASLQILAA
LYQLVAKPMQALEVLLLLRIVSERLKDHSKAAGSS
CHITQLLLTLGCPSYAQLHLEEAASSLKHLDQTTD
TYLLLSLTCDLLRSQLYWTHQKVTKGVSLLLSVLR
DPALQKSSKAWYLLRVQVLQLVAAYLSLPSNNLSH
SLWEQLCAQGWQTPEIALIDSHKLLRSIILLLMGS
DILSTQKAAVETSFLDYGENLVQKWQVLSEVLSCS
EKLVCHLGRLGSVSEAKAFCLEALKLTTKLQIPRQ
CALFLVLKGELELARNDIDLCQSDLQQVLFLLESC
TEFGGVTQHLDSVKKVHLQKGKQQAQVPCPPQLPE
EELFLRGPALELVATVAKEPGPIAPSTNSAPVLKT
KPQPIPNFLSHSPTCDCSLCASPVLTAVCLRWVLV
TAGVRLAMGHQAQGLDLLQVVLKGCPEAAERLTQA
LQASLNHKTPPSLVPSLLDEILAQAYTLLALEGLN
QPSNESLQKVLQSGLKFVAARIPHLEPWRASLLLI
WALTKLGGLSCCTTQLFASSWGWQPPLIKSVPGSE
PSKTQGQKRSGRGRQKLASAPLSLNNTSQKGLEGR
GLPCTPKPPDRIRQAGPHVPFTVFEEVCPTESKPE
VPQAPRVQQRVQTRLKVNFSDDSDLEDPVSAEAWL
AEEPKRRGTASRGRGRARKGLSLKTDAVVAPGSAP
GNPGLNGRSRRAKKVASRHCEERRPQRASDQARPG
PRIMETIPEEELTDNWRKMSFRILEGSDGEDSASG
GKTPAPGPEAASGEWRLLELDSSKKKLPSPCPDKE
SDKDLGPRLQLPSAPVATGLSTLDSICDSLSVAFR
GISHCPPSGLYAHLCRFLALCLGHRDPYATAFLVT
ESVSITCRHQLLTHLHRQLSKAQKHRGSLEIADQL
QGLSLQEMPGDVPLARIQRLFSFRALESGHFPQPE
KESFQERLALIPSGVTVCVLALATLQPGTVGNTLL
LTRLEKDSPPVSVQIPTGQNKLHLRSVLNEFDAIQ
KAQKENSSCTDKREWWTGRLALDHRMEVLIASLEK
SVLGCWKGLLLPSSEEPGPAQEASRLQELLQDCGW
KYPDRTLLKIMLSGAGALTPQDIQALAYGLCPTQP
ERAQELLNEAVGRLQGLTVPSNSHLVLVLDKDLQK
LPWESMPSLQALPVTRLPSFRFLLSYSIIKEYGAS
PVLSQGVDPRSTFYVLNPHNNLSSTEEQFRANFSS
EAGWRGVVGEVPRPEQVQEALTKHDLYIYAGHGAG
ARFLDGQAVLRLSCRAVALLFGCSSAALAVHGNLE
GAGIVLKYIMAGCPLFLGNLWDVTDRDIDRYTEAL
LQGWLGAGPGAPLLYYVNQARQAPRLKYLIGAAPI
AYGLPVSLR
SecurinΔ138-separase
SEQ ID NO: 5
MHWSHPQFEKGSAGSAAGSGAGWSHPQFEKGSSTA
SHLPLSGVPLMILDEERELEKLFQLGPPSPVKMPS
PPWESNLLQSPSSILSTLDVELPPVCCDIDIGGSG
GSGGGSGGGSGENLYFQGDYKDHDGDYKDHDIDYK
DDDDKSGPMRSFKRVNFGTLLSSQKEAEELLPDLK
EFLSNPPAGFPSSRSDAERRQACDAILRACNQQLT
AKLACPRHLGSLLELAELACDGYLVSTPQRPPLYL
ERILFVLLRNAAAQGSPEATLRLAQPLHACLVQCS
REAAPQDYEAVARGSFSLLWKGAEALLERRAAFAA
RLKALSFLVLLEDESTPCEVPHFASPTACRAVAAH
QLFDASGHGLNEADADFLDDLLSRHVIRALVGERG
SSSGLLSPQRALCLLELTLEHCRRFCWSRHHDKAI
SAVEKAHSYLRNTNLAPSLQLCQLGVKLLQVGEEG
PQAVAKLLIKASAVLSKSMEAPSPPLRALYESCQF
FLSGLERGTKRRYRLDAILSLFAFLGGYCSLLQQL
RDDGVYGGSSKQQQSFLQMYFQGLHLYTVVVYDFA
QGCQIVDLADLTQLVDSCKSTVVWMLEALEGLSGQ
ELTDHMGMTASYTSNLAYSFYSHKLYAEACAISEP
LCQHLGLVKPGTYPEVPPEKLHRCFRLQVESLKKL
GKQAQGCKMVILWLAALQPCSPEHMAEPVTFWVRV
KMDAARAGDKELQLKTLRDSLSGWDPETLALLLRE
ELQAYKAVRADTGQERFNIICDLLELSPEETPAGA
WARATHLVELAQVLCYHDFTQQTNCSALDAIREAL
QLLDSVRPEAQARDQLLDDKAQALLWLYICTLEAK
IQEGIERDRRAQAPGNLEEFEVNDLNYEDKLQEDR
FLYSNIAFNLAADAAQSKCLDQALALWKELLTKGQ
APAVRCLQQTAASLQILAALYQLVAKPMQALEVLL
LLRIVSERLKDHSKAAGSSCHITQLLLTLGCPSYA
QLHLEEAASSLKHLDQTTDTYLLLSLTCDLLRSQL
YWTHQKVTKGVSLLLSVLRDPALQKSSKAWYLLRV
QVLQLVAAYLSLPSNNLSHSLWEQLCAQGWQTPEI
ALIDSHKLLRSIILLLMGSDILSTQKAAVETSFLD
YGENLVQKWQVLSEVLSCSEKLVCHLGRLGSVSEA
KAFCLEALKLTTKLQIPRQCALFLVLKGELELARN
DIDLCQSDLQQVLFLLESCTEFGGVTQHLDSVKKV
HLQKGKQQAQVPCPPQLPEEELFLRGPALELVATV
AKEPGPIAPSTNSAPVLKTKPQPIPNFLSHSPTCD
CSLCASPVLTAVCLRWVLVTAGVRLAMGHQAQGLD
LLQVVLKGCPEAAERLTQALQASLNHKTPPSLVPS
LLDEILAQAYTLLALEGLNQPSNESLQKVLQSGLK
FVAARIPHLEPWRASLLLIWALTKLGGLSCCTTQL
FASSWGWQPPLIKSVPGSEPSKTQGQKRSGRGRQK
LASAPLSLNNTSQKGLEGRGLPCTPKPPDRIRQAG
PHVPFTVFEEVCPTESKPEVPQAPRVQQRVQTRLK
VNFSDDSDLEDPVSAEAWLAEEPKRRGTASRGRGR
ARKGLSLKTDAVVAPGSAPGNPGLNGRSRRAKKVA
SRHCEERRPQRASDQARPGPRIMETIPEEELTDNW
RKMSFRILEGSDGEDSASGGKTPAPGPEAASGEWR
LLELDSSKKKLPSPCPDKESDKDLGPRLQLPSAPV
ATGLSTLDSICDSLSVAFRGISHCPPSGLYAHLCR
FLALCLGHRDPYATAFLVTESVSITCRHQLLTHLH
RQLSKAQKHRGSLEIADQLQGLSLQEMPGDVPLAR
IQRLFSFRALESGHFPQPEKESFQERLALIPSGVT
VCVLALATLQPGTVGNTLLLTRLEKDSPPVSVQIP
TGQNKLHLRSVLNEFDAIQKAQKENSSCTDKREWW
TGRLALDHRMEVLIASLEKSVLGCWKGLLLPSSEE
PGPAQEASRLQELLQDCGWKYPDRTLLKIMLSGAG
ALTPQDIQALAYGLCPTQPERAQELLNEAVGRLQG
LTVPSNSHLVLVLDKDLQKLPWESMPSLQALPVTR
LPSFRFLLSYSIIKEYGASPVLSQGVDPRSTFYVL
NPHNNLSSTEEQFRANFSSEAGWRGVVGEVPRPEQ
VQEALTKHDLYIYAGHGAGARFLDGQAVLRLSCRA
VALLFGCSSAALAVHGNLEGAGIVLKYIMAGCPLF
LGNLWDVTDRDIDRYTEALLQGWLGAGPGAPLLYY
VNQARQAPRLKYLIGAAPIAYGLPVSLR
SecurinΔ160-separase
SEQ ID NO: 6
MHWSHPQFEKGSAGSAAGSGAGWSHPQFEKGSSTA
SQLGPPSPVKMPSPPWESNLLQSPSSILSTLDVEL
PPVCCDIDIGGSGGSGGGSGGGSGENLYFQGDYKD
HDGDYKDHDIDYKDDDDKSGPMRSFKRVNFGTLLS
SQKEAEELLPDLKEFLSNPPAGFPSSRSDAERRQA
CDAILRACNQQLTAKLACPRHLGSLLELAELACDG
YLVSTPQRPPLYLERILFVLLRNAAAQGSPEATLR
LAQPLHACLVQCSREAAPQDYEAVARGSFSLLWKG
AEALLERRAAFAARLKALSFLVLLEDESTPCEVPH
FASPTACRAVAAHQLFDASGHGLNEADADFLDDLL
SRHVIRALVGERGSSSGLLSPQRALCLLELTLEHC
RRFCWSRHHDKAISAVEKAHSYLRNTNLAPSLQLC
QLGVKLLQVGEEGPQAVAKLLIKASAVLSKSMEAP
SPPLRALYESCQFFLSGLERGTKRRYRLDAILSLF
AFLGGYCSLLQQLRDDGVYGGSSKQQQSFLQMYFQ
GLHLYTVVVYDFAQGCQIVDLADLTQLVDSCKSTV
VWMLEALEGLSGQELTDHMGMTASYTSNLAYSFYS
HKLYAEACAISEPLCQHLGLVKPGTYPEVPPEKLH
RCFRLQVESLKKLGKQAQGCKMVILWLAALQPCSP
EHMAEPVTFWVRVKMDAARAGDKELQLKTLRDSLS
GWDPETLALLLREELQAYKAVRADTGQERFNIICD
LLELSPEETPAGAWARATHLVELAQVLCYHDFTQQ
TNCSALDAIREALQLLDSVRPEAQARDQLLDDKAQ
ALLWLYICTLEAKIQEGIERDRRAQAPGNLEEFEV
NDLNYEDKLQEDRFLYSNIAFNLAADAAQSKCLDQ
ALALWKELLTKGQAPAVRCLQQTAASLQILAALYQ
LVAKPMQALEVLLLLRIVSERLKDHSKAAGSSCHI
TQLLLTLGCPSYAQLHLEEAASSLKHLDQTTDTYL
LLSLTCDLLRSQLYWTHQKVTKGVSLLLSVLRDPA
LQKSSKAWYLLRVQVLQLVAAYLSLPSNNLSHSLW
EQLCAQGWQTPEIALIDSHKLLRSIILLLMGSDIL
STQKAAVETSFLDYGENLVQKWQVLSEVLSCSEKL
VCHLGRLGSVSEAKAFCLEALKLTTKLQIPRQCAL
FLVLKGELELARNDIDLCQSDLQQVLFLLESCTEF
GGVTQHLDSVKKVHLQKGKQQAQVPCPPQLPEEEL
FLRGPALELVATVAKEPGPIAPSTNSAPVLKTKPQ
PIPNFLSHSPTCDCSLCASPVLTAVCLRWVLVTAG
VRLAMGHQAQGLDLLQVVLKGCPEAAERLTQALQA
SLNHKTPPSLVPSLLDEILAQAYTLLALEGLNQPS
NESLQKVLQSGLKFVAARIPHLEPWRASLLLIWAL
TKLGGLSCCTTQLFASSWGWQPPLIKSVPGSEPSK
TQGQKRSGRGRQKLASAPLSLNNTSQKGLEGRGLP
CTPKPPDRIRQAGPHVPFTVFEEVCPTESKPEVPQ
APRVQQRVQTRLKVNFSDDSDLEDPVSAEAWLAEE
PKRRGTASRGRGRARKGLSLKTDAVVAPGSAPGNP
GLNGRSRRAKKVASRHCEERRPQRASDQARPGPRI
METIPEEELTDNWRKMSFRILEGSDGEDSASGGKT
PAPGPEAASGEWRLLELDSSKKKLPSPCPDKESDK
DLGPRLQLPSAPVATGLSTLDSICDSLSVAFRGIS
HCPPSGLYAHLCRFLALCLGHRDPYATAFLVTESV
SITCRHQLLTHLHRQLSKAQKHRGSLEIADQLQGL
SLQEMPGDVPLARIQRLFSFRALESGHFPQPEKES
FQERLALIPSGVTVCVLALATLQPGTVGNTLLLTR
LEKDSPPVSVQIPTGQNKLHLRSVLNEFDAIQKAQ
KENSSCTDKREWWTGRLALDHRMEVLIASLEKSVL
GCWKGLLLPSSEEPGPAQEASRLQELLQDCGWKYP
DRTLLKIMLSGAGALTPQDIQALAYGLCPTQPERA
QELLNEAVGRLQGLTVPSNSHLVLVLDKDLQKLPW
ESMPSLQALPVTRLPSFRFLLSYSIIKEYGASPVL
SQGVDPRSTFYVLNPHNNLSSTEEQFRANFSSEAG
WRGVVGEVPRPEQVQEALTKHDLYIYAGHGAGARF
LDGQAVLRLSCRAVALLFGCSSAALAVHGNLEGAG
IVLKYIMAGCPLFLGNLWDVTDRDIDRYTEALLQG
WLGAGPGAPLLYYVNQARQAPRLKYLIGAAPIAYG
LPVSLR
Mouse (Mus musculus) separase
SEQ ID NO: 7
MRNFKGVNFATLLCSKEETQQLLPDLKEFLSRSRT
DFPSSRTDAERRQICDTILRACTQQLTAKLDCPGH
LRSILDLAELACDGYLLSTPQRPPLYLERILFILL
RNGSTQGSPDTVLRLAQPLHACLVQNSGEAAPQDY
EAVTRGSFSLFWKGAEALLERRAAFSTRLNALSFL
VLLEDGSVPCEVPHFASPTACRLVAAYQLYDATGQ
GLDEADADFLYEVLSRHLIRVLVGEGGSSPGPLSP
QRALCLLEITLEHCRRLCWNHHHRQAARAVERARN
HLEKTSVAPSLQLCQMGVELLEAVEERPGAVAQLL
RKAAAVLINSIEAPSPPLRALYDSCQFFLSGLERG
IRRHCGLDAILSLFAFLGGYSSLVRHLREVSEASS
KQQQCLLQMHFQGFHLFTGIVYDFAQGCQATELAQ
LVDGCRSAAVWMLEALEGLSGGELADYLSMTASYT
SNLAYSFFSQKLYEEACVISEPVCQHLGSATSGAC
PEVPPEKLHRCFRLHVESLKKLGKQAQGCKMVTLW
LAALKPYSLEHMVEPVTFWVRVKMDASRAGDKELQ
LQTLRDSLSCWDPETQSLLLREELRAYKSVRADTG
QERFNIICDLLELSPEETAAGAWARATYLVELAQV
LCYHNFTQQTNCSALDAVQEALQLLESVSPEAQEQ
DRLLDDKAQALLWLYICTLEAKMQEGIERDRRAQA
PSNLEEFEVNDLNYEDKLQEDRFLYSSIAFNLAAD
AAQSKCLDQALTLWKEVLTKGRAPAVRCLQQTAAS
LQILAAVYQLVAKPLQALETLLLLQIVSKRLQDHA
KAASSSCQLTQLLLNLGCPSYAQLYLEEAESSLRS
LDQTSDACQLLSLTCALLGSQLCWACQKVTAGVSL
LLSVLRDPALQKSSKAWYLLRVQALQVLAFYLSLS
SNLLSSALREQLWDQGWQTPETALIDAHKLLRSII
ILLMGSDVLSIQKAATESPFLDYGENLVQKWQVLT
EVLTCSERLVGRLGRLGNVSEAKAFCLEALKLTTK
LQIPRQCALFLVLKGELELARGDIDLCQSDLQQVL
FLLESSTEFGVVTQHPDSVKKVHTQKGKHKAQGPC
FPPLSEEEPFLKGPALELVDTVLNEPGPIQSSVNS
SPVLKTKPPPNPGFLSHLPSCDCLLCASPALSAVC
LRWVLVTAGVRLATGHKAQGLDLLQAVLTRCPAAT
KRFTQSLQASLNHRTTPSCVPSLFDEIMAQVYTHL
ALEFLNQTSEKSLGKVLASGLKFVATRIQSLEIWR
AHLLLVQALAKLAHFSCCTSELFASSWGWHPPLVK
SLPVLEPAKIRRQKCSGRGRRRIASVPPPLHNSSQ
KGLEEEGPPCTPKPPGRARQAGPRVPFTIFEEVHP
TKSKLQVPLAPRVHRRAQTRLKVIFSDDSDLEDLV
SADTQLVEEPKRRGTASRTRGQTRKGRSLKTDAVV
AIESTPGHSSVSGRTRRARKVASRNCEEESPKAPL
CVWASQGPEIMRSIPEEEPVDNHLEKSFEILRGSD
GEDSASGEKAAAADTGLPVGECEVLRRDSSKAERP
VLYSDTEANSDPSPWLPPFSVPAPIDLSTLDSISD
SLSIAFRGVSHCPPSGLYAHLCRFLALCLGHRDPY
ATAFLVAESISITCRHQLLTHLHRQLSKAQKQQES
PELAEHLQRLDLKERPGGVPLARIQRLFSFKALGS
GCFPQAEKESFQERLALIPSGVTVCVLALATLQPG
TLSNTLLLTRLEKDNPPITVKIPTAQNKLPLSAVL
KEFDAIQKDQKENSSCTEKRVWWTGRLALDQRMEA
LITALEEQVLGCWRGLLLPCSADPSLAQEASKLQE
LLRECGWEYPDSTLLKVILSGARILTSQDVQALAC
GLCPAQPDRAQVLLSEAVGQVQSQEAPRSQHLVLV
LDKDLQKLPWESTPILQAQPVTRLPSFRFLLSYTV
TKEAGASSVLSQGVDPQNTFYVLNPHSNLSSTEER
FRASFSSETGWKGVIGEVPSLDQVQAALTERDLYI
YAGHGAGARFLDGQAVLRLSCRAVALLFGCSSAAL
AVHGNLEGAGIVLKYIMAGCPLFLGNLWDVTDRDI
DRYTEALLQGWLGAGPGAPFLYYASQARQAPRLKY
LIGAAPVAYGLPISLQTP
Nematode
(Caenorhabditis elegans) separase
SEQ ID NO: 8
MKITNKSVDKQHIEKLDELRKNVSCTVIGFAEQTA
ELQQEISELFIAEFGVNGPIDMNSLSKLARITSYY
ASSEYFQGLAKYQRTACKMFITWQT
LRKEAMECRSKDREIFASIPAKLCFFYFYNGELCR
AVVCLLDYIDLSDDTLAKEAALRWLMFLGETELIE
KKLKTWKMDKSSKDMFSATEFAMNYLKKSEYRVEM
LEKLMKLRDKVKSDPTRSFSRYELASYVSWLCSTL
SNVPVGSALRECEFPDRVSHIQEAALKSDSLVRNR
IPGLASSQFDNSVNASIWPFLDGHQEDSNYYVHIG
STIAWHFEMRRECALVNVTTAQTRDSMSAMILNLR
VALKSASFFRVLQTTNTLAYYSSIIEEAGSEKNAK
LMRVSCVNLLSSNPIIVRCSTPKETGATSRAHTPM
AGSSVSEKQNTMRPDLADLLGDLELLDEQSFHPIT
RSCVCNVCTIYPLHSSFAAEYMMSYAIHSDFSQLS
IKHFNDEFARIRERGMSSQVLMHRDSSVRPRPNII
QNEIFGMCVIRWLTKKLDSKESADEDTMEIFNNAL
KIVRYLQQRTTDMILAVTQLGRQLEFPMECNYSWM
RPTIRKPRVKATIDCAVDILRAVSPFGRRPKVEKL
EKNLQPFDKERFEKVRLAMRNEMNHYGHILYREWR
CRLFAYVGRTSRDPWEAAYAWAESTQIGARNAVQS
RLEKCKRGLVTMSGHDRFKTCVQSMPDEMTLVQIA
MADDKTIYLVKLHADRDPIIMPLAHYSQAVELMDK
FTFLLDEDEMIAKYPGDITPEEFWKRRKIVDGRMM
TFVDEVQKHFLGVAASLLMPSGQLGPKAAELAIKI
HKLSKGGLLLGEAKEMVYQSKLMDAKSWEALILRF
CEMRTTDEKFKSFLPLMHRNSVEVMNQDDSIVTEK
KYTYLVICPHLSQFCWERLPIFDEYPYVGRQVSIH
STFSQLEAMKSQEKQIPLQIDVQNAYYILDPDNNL
GETQKRMVEYINKFNWEGTVGSAPKSNEISAALSQ
RDAFFFIGHGSGSSVMPRSVLKQSTCNAISLLMGC
GSVRTIPQALGFDGKTAILDYAMAKCPLIVGCLWT
VTDGEIDRFLIRMIDDCFEDSKSLTGIDKLRQLSE
AMHEARSKARLKYLTGAAVVMYGLPVVAKQTTPFV
EKDQRNLPQTPKTSARTSMRMETVPKTPKQEFVTS
KSVPMTPIFSNNENKSPSRARMPSRVLKTPRQVKT
FQEEDDEAPKRSTTRQLKPLVAPPIPATPTTRTTR
SSARTPSRSRNL
Budding yeast
(Saccharomyces cerevisiae) separase
SEQ ID NO: 9
MMVKQEEPLNEISPNTPMTSKSYLLNDTLSKVHHS
GQTRPLTSVLSGDASSNSIGILAMHNNIIRDFTKI
ASNNIDLAIEDITTVDHSLNSIYSLLKSHHMWGHI
NSTVKQHLMIIVKLINNNALGLASSEIIFLFNETN
LFQAHSLKNILLADFSTWNDYYLSNLKILALQIIL
KRKLVDEYLPHILELFSHDKRYLLKDPNLKAHALT
KIVLSFFSVTTSCKVLFGLKFLQYIKQFKLPFKKF
ISNITVECFSKNLLHKNYLEMGPNKIYLNSFYLSY
SMLYDGLDKIMLLDILSYEETTEVQRAIKSKKEFN
EYCNMSENRLLWSCISVDDLNVILENATNFLQNKG
KHISATLKCLVCLWSTIRLEGLPKNKDILRQFDCT
VIYINSNIKSINDESAAALLSELLGVLSEICIDYK
EPKRLSNIISVLFNASVLFKSHSFLLKTANLEISN
VLISNDSKTSHRTILKFEKFISSAQSAQKKIEIFS
CLFNVYCMLRNDTLSFVFDFCQNAFIHCFTRLKIT
KFIEFSNSSEIMLSVLYGNSSIENIPSENWSQLSR
MIFCSLRGIFDLDPLELNNTFDKLHLLNKYELLIR
IVYLLNLDMSKHLTTNLSKITKLYINKWLQKSDEK
AERISSFEMDFVKMLLCYLNFNNFDKLSIELSLCI
KSKEKYYSSIVPYADNYLLEAYLSLYMIDDALMMK
NQLQKTMNLSTAKIEQALLHASSLINVHLWDSDLT
AFQIYFGKTLPAMKPELFDINNDHNLPMSLYIKVI
LLNIKIFNESAKLNIKAGNVISAVIDCRKAQNLAL
SLLKKKNKLSQGSRLALLKSLSFSFFQLIKIHIRI
GSARDCEFYSKELSRIISDLEEPIIVYRCLHFLHR
YYMITEQTCLQNITLGKANKAFDYLDAEADITSLT
MFLYDNKEFVKLEQSLVLYFGDQLEKTFLPNLWKL
HLGKDIDDSICLSEYMPKNVINRVHNMWQKVMSQL
EEDPFFKGMFESTLGIPSSLPVIPSTMPNNILKTP
SKHSTGLKLCDSPRSSSMTPRGKNIRQKFDRIAAI
SKLKQMKELLESLKLDTLDNHELSKISSLSSLTLT
ILSNITSIHNAESSLITNFSLTDLPRHMPLLFDKV
LNNIDNKNYREFRVSSLIAPNNISTITESIRVSAA
QKDLMESNLNINVITIDFCPITGNLLLSKLEPRRK
RRTHLRLPLIRSNSRDLDEVHLSFPEATKKLLSII
NESNQTTSVEVTNKIKTREERKSWWTTRYDLDKRM
QQLLNNIENSWFNGVQGFFSPEVVDNSLFEKFKDK
FYEILHQNLPSRKLYGNPAMFIKVEDWVIELFLKL
NPQEIDFLSKMEDLIYFVLDILLFHGEENAYDEID
FSMLHVQLEEQIKKYRATMTTNSIFHTFLVVSSSC
HLFPWECLSFLKDLSITRVPSYVCLNKLLSRFHYQ
LPLQVTIEDNISMILNPNGDLSRTESKFKGMFQKI
IDAKPSSQLVMNEKPEEETLLKMLQNSNLFVYIGH
GGGEQYVRSKEIKKCTKIAPSFLLGCSSAAMKYYG
KLEPTGTIYTYLLGGCPMVLGNLWDVTDKDIDKFS
EELFEKMGFRCNTDDLNGNSLSVSYAVSKSRGVCH
LRYLNGAAPVIYGLPIKFVS
Fission yeast
(Schizosaccharomyces pombe) separase
SEQ ID NO: 10
MSTRSIVTSKVSWTPEKFISALSYPEHCSITLVKR
LKASVKLKDLKQNISRDAPSWTFEHLFVAFKCAVS
NLAKQWAELSTTDKEKTRRMFCTPSRLNTAHRPEV
FYLLECCTYILEQMQVVTKNTSHLYDCIRSGVSIC
NRLLDMEIFEPAISLLMKTHKNLIILLTYRDHDAI
PTATLLNPTLDVSEIQLESCLFVPMVPASYFLNIG
TIVVTFQLNVLRCLSLSQINGLSLNTINNLQSEDG
PFQWIERSFPSQVQLANSRREILARLLTRFSMIQN
NALQSFKLLILSIALWLNILSSQRADDKEFDVNQL
ETRILQLFSKVVQLCKSEDIEGSILNKDMTQLHHL
LENLSKESRLHILLQLSQLYYKYNDFQLSAAYVIR
GYSLSFEDISFKLKFLLFSFRLSIRONSICFPFNL
IQELSSLQQLFVENALPYSEALHLLDSIERSFRLF
NDSTVFDDTVFALNISEILSWILSSVVRDILVEDE
LLNLQLKIRKFLMFTFHIIRSFSELTKFQSSLEGC
LNLAAYYEDAEFPQKLSNHLYNLCVKSSNVNYARE
CISLSIKIAVSHKLTNDETYLLKILKNFQLRYHDS
LQLQEKCDVLHTTFNQLDLYVGTTSVGKSSVLDNI
LKRIFNSLTSINDSNIEKLLESISYSLLKLFFKCA
NEGSRYNASAALSFKLSLMLHEKEEVLLLKTNVSC
VLANHGYNDIKFEEMVLCVIKGDQNLLEHNSNNNA
KLALNESLLCSWENLLCYRRAEDDSRILTIIESWT
IFISRFSSVISRCSFTDFEINSILNFFFCFLHTVE
PSGKLTFELAFLEIFYELFNCLLHLQFSKYLVIIG
TLLSDKYMTLGFSGKAHLFYTKCYSYLRQCKSSPF
INFWNVSYGKYLILTGNTDKGILQLKKYSLSSEED
FNSNGLSRTVSLNLLLYERIQLSDALFQLGYTTVS
LGFIMQNLKVIKGLFSKSSKEHFNGGKYITWRLFA
VSAHSNVCAARIYEHMGQAREAEFFYRQACSISEK
MPFSCFSATFQLRLCSLLTRAGKLEKGEKILFDLT
EAMKSTDTYHKLLWNYGAAEVCATKSELDGAICHY
SECVKLLEIIKSEYYLFFNRNREKSLTKGIKRLSL
SSQPTFVTESNTTEFDDWSILQNTAANLLRLISMF
ELKRGNLEIAKALMTDSTKCSIASFFNIVSANILK
SKLIVCEADSTLFGDPVLRTLPDSVISLPGISHKF
QKNQSKTKALGENTGFRKGSKRLDYLRERLKINLQ
NVRLSCEIIFSNAYERSSVCVCREVNELISYSTIM
QSALTTIGETTDVDSSSASFFLEIPKALGFHRRRE
AQKFRNQHKELHFSSLEQILNSRLSIPDVRTFQDN
FIDSLPSIWNVVSITINNSGEDLFISKIRKGHSPL
IFRLPLQRHNSRDADEEILVFTKAQTELFRIISKS
NQMAQNGKHYTRREDKETWWKERRHLDQCLQQLLE
NIEISWLGGFKGIFNPHKIDTSLFAKFSSQFQNII
AKNFNMDKKTPVPTLSPEILELFITLGKPGYEGYE
QLLEDLIYFILDIFQFRGLHFAYDEIDTDQLSMDL
QDALNAYFNNYVSEENRSHTVLVLDKSVHQFPWES
LPCLNRQSVSRVPSLSILRDILSQSFVVNGEYVEV
RKEAGSYILNPSLDLKHTQEMFEHKLVEGGWKGLI
ASQPSNRDFIKMLSGNDFFLYFGHGGGEQYTTSYD
LATLKRCAVTILMGCSSGALYECGSFEPWGTPLDY
LSAGCPTLVANLWDVTDKDIDRFSLKMLESWGLFE
NKAPFVNSTSICTAVSESRSCCHLRYLNGAAPVIY
GIPAYIIP
Mouse (Mus musculus) securin
SEQ ID NO: 11
MATLIFVDKDNEEPGRRLASKDGLKLGTGVKALDG
KLQVSTPRVGKVFNAPAVPKASRKALGTVNRVAEK
PMKTGKPLQPKQPTLTGKKITEKSTKTQSSVPAPD
DAYPEIEKFFPFNPLDFESFDLPEEHQISLLPLNG
VPLMTLNEERGLEKLLHLGPPSPLKTPFLSWESDP
LYSPPSALSTLDVELPPVCYDADI
Nematode (Caenorhabditis elegans)
securin
SEQ ID NO: 12
MEDLNFEERGSTQIPASLQQHFSAKLGRQNELEKT
PSRGGLGLVVNSSKTPGGKSLQSLASACKVPPSTK
KNTIPIAFECYEDETDDQIADVATIKKTEKHPCSP
IDTANRCETFDSLAADIEDDMLNLEDQDVVLSEDR
PYGDVIDPAESEAEALAELGVEEWDSYPPIDPASR
IGDDFNYVLRTEDFAEEGDVKLEETRHRTVIADID
EVKMSKAERNELFSMLADDLDSYDLLAEEANLPL
Budding yeast
(Saccharomyces cerevisiae) securin
SEQ ID NO: 13
MMPANEDKENNIVYTGNESSGINFPQTPAHLLKRS
HSNILKPPVRLDQLKRDANSNNGNTLKYIQGGKEV
SPTKRLHTHAQQQGRLPLAAKDNNRSKSFIFPETS
NQSKDADLPQLQNTLSIRKNDQLRKLSQISRSRSR
ANHNDLLSNSRKLQKYGSVLGYNALPKMKSLVLKD
LADSGKNEESSDDDEGNEDSESKLGKKLQSALLKQ
DSSDGENELNGGLGLFNEQGGLQQLIKNSTKNEQK
TKNDKSDKTDDYDIEIAPQRQEPLPYVPEGYSPFQ
QDDIEKLKTFNSPYKLDLEDEDDTPDKVDLLPLEQ
IDEEGEKDETECITRNQEEGAALPLLSKNFKEVAA
VPTMELVYSEEGLDPEELEDLVT
Fission yeast
(Schizosaccharomyces pombe) securin
SEQ ID NO: 14
MLPRTMFSYGKENAFPVTPISNRNGTKGAGSKRAP
LGSTKQSNAPSSVTVPRTVLGGKSTNISKFISAPS
TKKMSPMDISMDSPTILEPNSQGISRSAVQERSKR
LSASPRRSSLTDTPLPNELEEDIEYMPPPVHLDPI
QSLGFDDVAIDCETLDPWPSMQNKATSVTIRNTPA
SDFHVYKEFSDDDPIQFPLLSVDGDSPLTEKDTNL
TTPATLKASDQQRKVLEKPSVSKQSSSRTRLSTVY
RTKLASGKSIPRPLSHKLTRPRVTASGNSRRRPLS
RSIHSLSSSRIDFSSLDTGLL
SEQ ID NO: 15
GGGGS
SEQ ID NO: 16
GGGGSGGGGS
SEQ ID NO: 17
GGGGSGGGGSGGGGS
SEQ ID NO: 18
GGGGSGGGGSGGGGSGGGGS
SEQ ID NO: 19
GGGGGG
SEQ ID NO: 20
GGGGGGGG
SEQ ID NO: 21
GGSGGSGGGSGGGSG
SEQ ID NO: 22
HHHHHH
SEQ ID NO: 23
DYKDDDDK
SEQ ID NO: 24
TNTAKILNFGR
SEQ ID NO: 25
DDREIMREGS

What is claimed is:

1. A polypeptide construct comprising a securin fused to a separase.

2. The polypeptide construct of claim 1, wherein the separase comprises an amino acid sequence having at least 90% identity to SEQ ID NO:1.

3. The polypeptide construct of claim 1 or claim 2, wherein the securin is a full-length securin or a truncated securin.

4. The polypeptide construct of any one of claims 1-3, wherein the securin comprises an amino acid sequence having at least 90% identity to positions 93-202 of SEQ ID NO:2.

5. The polypeptide construct of any one of claims 1-4, wherein the securin is fused to the separase via a linker peptide.

6. The polypeptide construct of claim 5, wherein the linker peptide comprises a protease recognition site.

7. The polypeptide construct of claim 6, wherein the protease is a site-specific endopeptidase.

8. The polypeptide construct of claim 6, wherein the protease is Tobacco Etch Virus (TEV) protease.

9. The polypeptide construct of any one of claims 5-8, wherein the linker peptide comprises an affinity tag.

10. The polypeptide construct of any one of claims 1-9, further comprising an unfoldase recognition site linked to the securin.

11. The polypeptide construct of claim 10, wherein the unfoldase recognition site is linked to the securin via an affinity tag.

12. The polypeptide construct according to claim 10 or claim 11, wherein the unfoldase is E. coli ClpX.

13. The polypeptide construct of any one of claims 1-12, wherein the securin consists of an amino acid sequence having at least 90% identity to positions 160-202 of SEQ ID NO:2.

14. The polypeptide construct of any one of claims 1-12, wherein the securin consists of an amino acid sequence having at least 90% identity to positions 138-202 of SEQ ID NO:2.

15. The polypeptide construct of any one of claims 1-12, wherein the securin consists of an amino acid sequence having at least 90% identity to positions 127-202 of SEQ ID NO:2.

16. The polypeptide construct of claim 1 comprising an amino acid sequence according to SEQ ID NO:3, SEQ ID NO. 4, SEQ ID NO. 5, or SEQ ID NO. 6.

17. A polypeptide construct comprising a securin linked to an unfoldase recognition site.

18. A nucleic acid encoding a polypeptide construct according to any one of claims 1-17.

19. A vector comprising a nucleic acid according to claim 18.

20. A host cell comprising a nucleic acid according to claim 18 or a vector according to claim 19.

21. A mixture comprising a polypeptide construct according to any one of claims 1-16 and one or more test substances.

22. The mixture of claim 29, wherein the test substance is an organic small-molecule separase inhibitor candidate.

23. A method for identifying a separase modulator compound, the method comprising:
(i) measuring a level or rate of peptide substrate cleavage by a polypeptide construct in the presence of a candidate compound, wherein the polypeptide construct comprises a securin fused to a separase;
(ii) measuring a level or rate of peptide substrate cleavage by the polypeptide construct in the absence of the candidate compound; and
(iii) identifying the candidate compound as a separase modulator compound when the level or rate of peptide substrate cleavage in step (i) is higher or lower than the level or rate of peptide substrate cleavage in step (ii).

24. The method of claim 23, wherein the peptide substrate comprises an LPE motif.

25. The method of claim 23 or claim 24, wherein the polypeptide construct is a construct according to any one of claims 1-16.

26. The method of any one of claims 23-25, wherein the level or rate of peptide substrate cleavage in step (i) is lower than the level or rate of the peptide substrate cleavage in step (ii), and the candidate compound is identified as a separase inhibitor.

27. A method for obtaining an active separase fusion protein, the method comprising expressing a polypeptide comprising a truncated securin fused to a separase, thereby obtaining the active separase fusion protein.

28. A method for obtaining an active separase, the method comprising:
(a) co-expressing a separase and a securin, wherein the securin is linked to an unfoldase recognition site; and
(b) combining the co-expressed separase and securin with an unfoldase-peptidase complex;
thereby removing the securin and obtaining the active separase.

29. A method for obtaining an active separase, the method comprising:
(1) expressing a polypeptide comprising a securin fused to a separase; and
(2) removing the securin from the expressed polypeptide, thereby obtaining the active separase;
wherein the active separase is substantially free of the securin.

30. The method of claim 29, wherein the securin is fused to the separase via a linker comprising a protease recognition site, and wherein removing the securin from the expressed polypeptide comprises cleaving the securin from the separase at the protease recognition site.

31. The method of claim 29 or claim 30, wherein the polypeptide further comprises an unfoldase recognition site linked to the securin, and wherein removing the securin from the expressed polypeptide comprises combining the expressed polypeptide with an unfoldase-peptidase complex.

32. An isolated active separase, which is substantially free of securin.

33. The isolated active separase of claim 32, which is obtained according to the method of any one of claims 29-31.


SEQUENCE LISTING (37 sequences)

SEQ ID NO: 1 - protein, 2120 aa, Homo sapiens
MRSFKRVNFGTLLSSQKEAEELLPDLKEFLSNPPAGFPSSRSDAERRQACDAILRACNQQLTAKLACPRHLGSLLELAELACDGYLVSTPQRPPLYLERILFVLLRNAAAQGSPEATLRLAQPLHACLVQCSREAAPQDYEAVARGSFSLLWKGAEALLERRAAFAARLKALSFLVLLEDESTPCEVPHFASPTACRAVAAHQLFDASGHGLNEADADFLDDLLSRHVIRALVGERGSSSGLLSPQRALCLLELTLEHCRRFCWSRHHDKAISAVEKAHSYLRNTNLAPSLQLCQLGVKLLQVGEEGPQAVAKLLIKASAVLSKSMEAPSPPLRALYESCQFFLSGLERGTKRRYRLDAILSLFAFLGGYCSLLQQLRDDGVYGGSSKQQQSFLQMYFQGLHLYTVVVYDFAQGCQIVDLADLTQLVDSCKSTVVWMLEALEGLSGQELTDHMGMTASYTSNLAYSFYSHKLYAEACAISEPLCQHLGLVKPGTYPEVPPEKLHRCFRLQVESLKKLGKQAQGCKMVILWLAALQPCSPEHMAEPVTFWVRVKMDAARAGDKELQLKTLRDSLSGWDPETLALLLREELQAYKAVRADTGQERFNIICDLLELSPEETPAGAWARATHLVELAQVLCYHDFTQQTNCSALDAIREALQLLDSVRPEAQARDQLLDDKAQALLWLYICTLEAKIQEGIERDRRAQAPGNLEEFEVNDLNYEDKLQEDRFLYSNIAFNLAADAAQSKCLDQALALWKELLTKGQAPAVRCLQQTAASLQILAALYQLVAKPMQALEVLLLLRIVSERLKDHSKAAGSSCHITQLLLTLGCPSYAQLHLEEAASSLKHLDQTTDTYLLLSLTCDLLRSQLYWTHQKVTKGVSLLLSVLRDPALQKSSKAWYLLRVQVLQLVAAYLSLPSNNLSHSLWEQLCAQGWQTPEIALIDSHKLLRSIILLLMGSDILSTQKAAVETSFLDYGENLVQKWQVLSEVLSCSEKLVCHLGRLGSVSEAKAFCLEALKLTTKLQIPRQCALFLVLKGELELARNDIDLCQSDLQQVLFLLESCTEFGGVTQHLDSVKKVHLQKGKQQAQVPCPPQLPEEELFLRGPALELVATVAKEPGPIAPSTNSAPVLKTKPQPIPNFLSHSPTCDCSLCASPVLTAVCLRWVLVTAGVRLAMGHQAQGLDLLQVVLKGCPEAAERLTQALQASLNHKTPPSLVPSLLDEILAQAYTLLALEGLNQPSNESLQKVLQSGLKFVAARIPHLEPWRASLLLIWALTKLGGLSCCTTQLFASSWGWQPPLIKSVPGSEPSKTQGQKRSGRGRQKLASAPLSLNNTSQKGLEGRGLPCTPKPPDRIRQAGPHVPFTVFEEVCPTESKPEVPQAPRVQQRVQTRLKVNFSDDSDLEDPVSAEAWLAEEPKRRGTASRGRGRARKGLSLKTDAVVAPGSAPGNPGLNGRSRRAKKVASRHCEERRPQRASDQARPGPRIMETIPEEELTDNWRKMSFRILEGSDGEDSASGGKTPAPGPEAASGEWRLLELDSSKKKLPSPCPDKESDKDLGPRLQLPSAPVATGLSTLDSICDSLSVAFRGISHCPPSGLYAHLCRFLALCLGHRDPYATAFLVTESVSITCRHQLLTHLHRQLSKAQKHRGSLEIADQLQGLSLQEMPGDVPLARIQRLFSFRALESGHFPQPEKESFQERLALIPSGVTVCVLALATLQPGTVGNTLLLTRLEKDSPPVSVQIPTGQNKLHLRSVLNEFDAIQKAQKENSSCTDKREWWTGRLALDHRMEVLIASLEKSVLGCWKGLLLPSSEEPGPAQEASRLQELLQDCGWKYPDRTLLKIMLSGAGALTPQDIQALAYGLCPTQPERAQELLNEAVGRLQGLTVPSNSHLVLVLDKDLQKLPWESMPSLQALPVTRLPSFRFLLSYSIIKEYGASPVLSQGVDPRSTFYVLNPHNNLSSTEEQFRANFSSEAGWRGVVGEVPRPEQVQEALTKHDLYIYAGHGAGARFLDGQAVLRLSCRAVALLFGCSSAALAVHGNLEGAGIVLKYIMAGCPLFLGNLWDVTDRDIDRYTEALLQGWLGAGPGAPLLYYVNQARQAPRLKYLIGAAPIAYGLPVSLR

SEQ ID NO: 2 - protein, 202 aa, Homo sapiens
MATLIYVDKENGEPGTRVVAKDGLKLGSGPSIKALDGRSQVSTPRFGKTFDAPPALPKATRKALGTVNRATEKSVKTKGPLKQKQPSFSAKKMTEKTVKAKSSVPASDDAYPEIEKFFPFNPLDFESFDLPEEHQIAHLPLSGVPLMILDEERELEKLFQLGPPSPVKMPSPPWESNLLQSPSSILSTLDVELPPVCCDIDI

SEQ ID NO: 3 - protein, 2321 aa, Artificial Sequence
  Description of Artificial Sequence Synthetic polypeptide
MTNTAKILNFGRWSHPQFEKGSAGSAAGSGAGWSHPQFEKGSASMTEKTVKAKSSVPASDDAYPEIEKFFPFNPLDFESFDLPEEHQIAHLPLSGVPLMILDEERELEKLFQLGPPSPVKMPSPPWESNLLQSPSSILSTLDVELPPVCCDIDIGGSGGSGGGSGGGSGENLYFQGDYKDHDGDYKDHDIDYKDDDDKSGPMRSFKRVNFGTLLSSQKEAEELLPDLKEFLSNPPAGFPSSRSDAERRQACDAILRACNQQLTAKLACPRHLGSLLELAELACDGYLVSTPQRPPLYLERILFVLLRNAAAQGSPEATLRLAQPLHACLVQCSREAAPQDYEAVARGSFSLLWKGAEALLERRAAFAARLKALSFLVLLEDESTPCEVPHFASPTACRAVAAHQLFDASGHGLNEADADFLDDLLSRHVIRALVGERGSSSGLLSPQRALCLLELTLEHCRRFCWSRHHDKAISAVEKAHSYLRNTNLAPSLQLCQLGVKLLQVGEEGPQAVAKLLIKASAVLSKSMEAPSPPLRALYESCQFFLSGLERGTKRRYRLDAILSLFAFLGGYCSLLQQLRDDGVYGGSSKQQQSFLQMYFQGLHLYTVVVYDFAQGCQIVDLADLTQLVDSCKSTVVWMLEALEGLSGQELTDHMGMTASYTSNLAYSFYSHKLYAEACAISEPLCQHLGLVKPGTYPEVPPEKLHRCFRLQVESLKKLGKQAQGCKMVILWLAALQPCSPEHMAEPVTFWVRVKMDAARAGDKELQLKTLRDSLSGWDPETLALLLREELQAYKAVRADTGQERFNIICDLLELSPEETPAGAWARATHLVELAQVLCYHDFTQQTNCSALDAIREALQLLDSVRPEAQARDQLLDDKAQALLWLYICTLEAKIQEGIERDRRAQAPGNLEEFEVNDLNYEDKLQEDRFLYSNIAFNLAADAAQSKCLDQALALWKELLTKGQAPAVRCLQQTAASLQILAALYQLVAKPMQALEVLLLLRIVSERLKDHSKAAGSSCHITQLLLTLGCPSYAQLHLEEAASSLKHLDQTTDTYLLLSLTCDLLRSQLYWTHQKVTKGVSLLLSVLRDPALQKSSKAWYLLRVQVLQLVAAYLSLPSNNLSHSLWEQLCAQGWQTPEIALIDSHKLLRSIILLLMGSDILSTQKAAVETSFLDYGENLVQKWQVLSEVLSCSEKLVCHLGRLGSVSEAKAFCLEALKLTTKLQIPRQCALFLVLKGELELARNDIDLCQSDLQQVLFLLESCTEFGGVTQHLDSVKKVHLQKGKQQAQVPCPPQLPEEELFLRGPALELVATVAKEPGPIAPSTNSAPVLKTKPQPIPNFLSHSPTCDCSLCASPVLTAVCLRWVLVTAGVRLAMGHQAQGLDLLQVVLKGCPEAAERLTQALQASLNHKTPPSLVPSLLDEILAQAYTLLALEGLNQPSNESLQKVLQSGLKFVAARIPHLEPWRASLLLIWALTKLGGLSCCTTQLFASSWGWQPPLIKSVPGSEPSKTQGQKRSGRGRQKLASAPLSLNNTSQKGLEGRGLPCTPKPPDRIRQAGPHVPFTVFEEVCPTESKPEVPQAPRVQQRVQTRLKVNFSDDSDLEDPVSAEAWLAEEPKRRGTASRGRGRARKGLSLKTDAVVAPGSAPGNPGLNGRSRRAKKVASRHCEERRPQRASDQARPGPRIMETIPEEELTDNWRKMSFRILEGSDGEDSASGGKTPAPGPEAASGEWRLLELDSSKKKLPSPCPDKESDKDLGPRLQLPSAPVATGLSTLDSICDSLSVAFRGISHCPPSGLYAHLCRFLALCLGHRDPYATAFLVTESVSITCRHQLLTHLHRQLSKAQKHRGSLEIADQLQGLSLQEMPGDVPLARIQRLFSFRALESGHFPQPEKESFQERLALIPSGVTVCVLALATLQPGTVGNTLLLTRLEKDSPPVSVQIPTGQNKLHLRSVLNEFDAIQKAQKENSSCTDKREWWTGRLALDHRMEVLIASLEKSVLGCWKGLLLPSSEEPGPAQEASRLQELLQDCGWKYPDRTLLKIMLSGAGALTPQDIQALAYGLCPTQPERAQELLNEAVGRLQGLTVPSNSHLVLVLDKDLQKLPWESMPSLQALPVTRLPSFRFLLSYSIIKEYGASPVLSQGVDPRSTFYVLNPHNNLSSTEEQFRANFSSEAGWRGVVGEVPRPEQVQEALTKHDLYIYAGHGAGARFLDGQAVLRLSCRAVALLFGCSSAALAVHGNLEGAGIVLKYIMAGCPLFLGNLWDVTDRDIDRYTEALLQGWLGAGPGAPLLYYVNQARQAPRLKYLIGAAPIAYGLPVSLR

SEQ ID NO: 4 - protein, 2284 aa, Artificial Sequence
  Description of Artificial Sequence Synthetic polypeptide
MHWSHPQFEKGSAGSAAGSGAGWSHPQFEKGSSTASENLYFQGSFDLPEEHQIAHLPLSGVPLMILDEERELEKLFQLGPPSPVKMPSPPWESNLLQSPSSILSTLDVELPPVCCDIDIGGSGGSGGGSGGGSGGGSGGDYKDHDGDYKDHDIDYKDDDDKSGPMRSFKRVNFGTLLSSQKEAEELLPDLKEFLSNPPAGFPSSRSDAERRQACDAILRACNQQLTAKLACPRHLGSLLELAELACDGYLVSTPQRPPLYLERILFVLLRNAAAQGSPEATLRLAQPLHACLVQCSREAAPQDYEAVARGSFSLLWKGAEALLERRAAFAARLKALSFLVLLEDESTPCEVPHFASPTACRAVAAHQLFDASGHGLNEADADFLDDLLSRHVIRALVGERGSSSGLLSPQRALCLLELTLEHCRRFCWSRHHDKAISAVEKAHSYLRNTNLAPSLQLCQLGVKLLQVGEEGPQAVAKLLIKASAVLSKSMEAPSPPLRALYESCQFFLSGLERGTKRRYRLDAILSLFAFLGGYCSLLQQLRDDGVYGGSSKQQQSFLQMYFQGLHLYTVVVYDFAQGCQIVDLADLTQLVDSCKSTVVWMLEALEGLSGQELTDHMGMTASYTSNLAYSFYSHKLYAEACAISEPLCQHLGLVKPGTYPEVPPEKLHRCFRLQVESLKKLGKQAQGCKMVILWLAALQPCSPEHMAEPVTFWVRVKMDAARAGDKELQLKTLRDSLSGWDPETLALLLREELQAYKAVRADTGQERFNIICDLLELSPEETPAGAWARATHLVELAQVLCYHDFTQQTNCSALDAIREALQLLDSVRPEAQARDQLLDDKAQALLWLYICTLEAKIQEGIERDRRAQAPGNLEEFEVNDLNYEDKLQEDRFLYSNIAFNLAADAAQSKCLDQALALWKELLTKGQAPAVRCLQQTAASLQILAALYQLVAKPMQALEVLLLLRIVSERLKDHSKAAGSSCHITQLLLTLGCPSYAQLHLEEAASSLKHLDQTTDTYLLLSLTCDLLRSQLYWTHQKVTKGVSLLLSVLRDPALQKSSKAWYLLRVQVLQLVAAYLSLPSNNLSHSLWEQLCAQGWQTPEIALIDSHKLLRSIILLLMGSDILSTQKAAVETSFLDYGENLVQKWQVLSEVLSCSEKLVCHLGRLGSVSEAKAFCLEALKLTTKLQIPRQCALFLVLKGELELARNDIDLCQSDLQQVLFLLESCTEFGGVTQHLDSVKKVHLQKGKQQAQVPCPPQLPEEELFLRGPALELVATVAKEPGPIAPSTNSAPVLKTKPQPIPNFLSHSPTCDCSLCASPVLTAVCLRWVLVTAGVRLAMGHQAQGLDLLQVVLKGCPEAAERLTQALQASLNHKTPPSLVPSLLDEILAQAYTLLALEGLNQPSNESLQKVLQSGLKFVAARIPHLEPWRASLLLIWALTKLGGLSCCTTQLFASSWGWQPPLIKSVPGSEPSKTQGQKRSGRGRQKLASAPLSLNNTSQKGLEGRGLPCTPKPPDRIRQAGPHVPFTVFEEVCPTESKPEVPQAPRVQQRVQTRLKVNFSDDSDLEDPVSAEAWLAEEPKRRGTASRGRGRARKGLSLKTDAVVAPGSAPGNPGLNGRSRRAKKVASRHCEERRPQRASDQARPGPRIMETIPEEELTDNWRKMSFRILEGSDGEDSASGGKTPAPGPEAASGEWRLLELDSSKKKLPSPCPDKESDKDLGPRLQLPSAPVATGLSTLDSICDSLSVAFRGISHCPPSGLYAHLCRFLALCLGHRDPYATAFLVTESVSITCRHQLLTHLHRQLSKAQKHRGSLEIADQLQGLSLQEMPGDVPLARIQRLFSFRALESGHFPQPEKESFQERLALIPSGVTVCVLALATLQPGTVGNTLLLTRLEKDSPPVSVQIPTGQNKLHLRSVLNEFDAIQKAQKENSSCTDKREWWTGRLALDHRMEVLIASLEKSVLGCWKGLLLPSSEEPGPAQEASRLQELLQDCGWKYPDRTLLKIMLSGAGALTPQDIQALAYGLCPTQPERAQELLNEAVGRLQGLTVPSNSHLVLVLDKDLQKLPWESMPSLQALPVTRLPSFRFLLSYSIIKEYGASPVLSQGVDPRSTFYVLNPHNNLSSTEEQFRANFSSEAGWRGVVGEVPRPEQVQEALTKHDLYIYAGHGAGARFLDGQAVLRLSCRAVALLFGCSSAALAVHGNLEGAGIVLKYIMAGCPLFLGNLWDVTDRDIDRYTEALLQGWLGAGPGAPLLYYVNQARQAPRLKYLIGAAPIAYGLPVSLR

SEQ ID NO: 5 - protein, 2268 aa, Artificial Sequence
  Description of Artificial Sequence Synthetic polypeptide
MHWSHPQFEKGSAGSAAGSGAGWSHPQFEKGSSTASHLPLSGVPLMILDEERELEKLFQLGPPSPVKMPSPPWESNLLQSPSSILSTLDVELPPVCCDIDIGGSGGSGGGSGGGSGENLYFQGDYKDHDGDYKDHDIDYKDDDDKSGPMRSFKRVNFGTLLSSQKEAEELLPDLKEFLSNPPAGFPSSRSDAERRQACDAILRACNQQLTAKLACPRHLGSLLELAELACDGYLVSTPQRPPLYLERILFVLLRNAAAQGSPEATLRLAQPLHACLVQCSREAAPQDYEAVARGSFSLLWKGAEALLERRAAFAARLKALSFLVLLEDESTPCEVPHFASPTACRAVAAHQLFDASGHGLNEADADFLDDLLSRHVIRALVGERGSSSGLLSPQRALCLLELTLEHCRRFCWSRHHDKAISAVEKAHSYLRNTNLAPSLQLCQLGVKLLQVGEEGPQAVAKLLIKASAVLSKSMEAPSPPLRALYESCQFFLSGLERGTKRRYRLDAILSLFAFLGGYCSLLQQLRDDGVYGGSSKQQQSFLQMYFQGLHLYTVVVYDFAQGCQIVDLADLTQLVDSCKSTVVWMLEALEGLSGQELTDHMGMTASYTSNLAYSFYSHKLYAEACAISEPLCQHLGLVKPGTYPEVPPEKLHRCFRLQVESLKKLGKQAQGCKMVILWLAALQPCSPEHMAEPVTFWVRVKMDAARAGDKELQLKTLRDSLSGWDPETLALLLREELQAYKAVRADTGQERFNIICDLLELSPEETPAGAWARATHLVELAQVLCYHDFTQQTNCSALDAIREALQLLDSVRPEAQARDQLLDDKAQALLWLYICTLEAKIQEGIERDRRAQAPGNLEEFEVNDLNYEDKLQEDRFLYSNIAFNLAADAAQSKCLDQALALWKELLTKGQAPAVRCLQQTAASLQILAALYQLVAKPMQALEVLLLLRIVSERLKDHSKAAGSSCHITQLLLTLGCPSYAQLHLEEAASSLKHLDQTTDTYLLLSLTCDLLRSQLYWTHQKVTKGVSLLLSVLRDPALQKSSKAWYLLRVQVLQLVAAYLSLPSNNLSHSLWEQLCAQGWQTPEIALIDSHKLLRSIILLLMGSDILSTQKAAVETSFLDYGENLVQKWQVLSEVLSCSEKLVCHLGRLGSVSEAKAFCLEALKLTTKLQIPRQCALFLVLKGELELARNDIDLCQSDLQQVLFLLESCTEFGGVTQHLDSVKKVHLQKGKQQAQVPCPPQLPEEELFLRGPALELVATVAKEPGPIAPSTNSAPVLKTKPQPIPNFLSHSPTCDCSLCASPVLTAVCLRWVLVTAGVRLAMGHQAQGLDLLQVVLKGCPEAAERLTQALQASLNHKTPPSLVPSLLDEILAQAYTLLALEGLNQPSNESLQKVLQSGLKFVAARIPHLEPWRASLLLIWALTKLGGLSCCTTQLFASSWGWQPPLIKSVPGSEPSKTQGQKRSGRGRQKLASAPLSLNNTSQKGLEGRGLPCTPKPPDRIRQAGPHVPFTVFEEVCPTESKPEVPQAPRVQQRVQTRLKVNFSDDSDLEDPVSAEAWLAEEPKRRGTASRGRGRARKGLSLKTDAVVAPGSAPGNPGLNGRSRRAKKVASRHCEERRPQRASDQARPGPRIMETIPEEELTDNWRKMSFRILEGSDGEDSASGGKTPAPGPEAASGEWRLLELDSSKKKLPSPCPDKESDKDLGPRLQLPSAPVATGLSTLDSICDSLSVAFRGISHCPPSGLYAHLCRFLALCLGHRDPYATAFLVTESVSITCRHQLLTHLHRQLSKAQKHRGSLEIADQLQGLSLQEMPGDVPLARIQRLFSFRALESGHFPQPEKESFQERLALIPSGVTVCVLALATLQPGTVGNTLLLTRLEKDSPPVSVQIPTGQNKLHLRSVLNEFDAIQKAQKENSSCTDKREWWTGRLALDHRMEVLIASLEKSVLGCWKGLLLPSSEEPGPAQEASRLQELLQDCGWKYPDRTLLKIMLSGAGALTPQDIQALAYGLCPTQPERAQELLNEAVGRLQGLTVPSNSHLVLVLDKDLQKLPWESMPSLQALPVTRLPSFRFLLSYSIIKEYGASPVLSQGVDPRSTFYVLNPHNNLSSTEEQFRANFSSEAGWRGVVGEVPRPEQVQEALTKHDLYIYAGHGAGARFLDGQAVLRLSCRAVALLFGCSSAALAVHGNLEGAGIVLKYIMAGCPLFLGNLWDVTDRDIDRYTEALLQGWLGAGPGAPLLYYVNQARQAPRLKYLIGAAPIAYGLPVSLR

SEQ ID NO: 6 - protein, 2246 aa, Artificial Sequence
  Description of Artificial Sequence Synthetic polypeptide
MHWSHPQFEKGSAGSAAGSGAGWSHPQFEKGSSTASQLGPPSPVKMPSPPWESNLLQSPSSILSTLDVELPPVCCDIDIGGSGGSGGGSGGGSGENLYFQGDYKDHDGDYKDHDIDYKDDDDKSGPMRSFKRVNFGTLLSSQKEAEELLPDLKEFLSNPPAGFPSSRSDAERRQACDAILRACNQQLTAKLACPRHLGSLLELAELACDGYLVSTPQRPPLYLERILFVLLRNAAAQGSPEATLRLAQPLHACLVQCSREAAPQDYEAVARGSFSLLWKGAEALLERRAAFAARLKALSFLVLLEDESTPCEVPHFASPTACRAVAAHQLFDASGHGLNEADADFLDDLLSRHVIRALVGERGSSSGLLSPQRALCLLELTLEHCRRFCWSRHHDKAISAVEKAHSYLRNTNLAPSLQLCQLGVKLLQVGEEGPQAVAKLLIKASAVLSKSMEAPSPPLRALYESCQFFLSGLERGTKRRYRLDAILSLFAFLGGYCSLLQQLRDDGVYGGSSKQQQSFLQMYFQGLHLYTVVVYDFAQGCQIVDLADLTQLVDSCKSTVVWMLEALEGLSGQELTDHMGMTASYTSNLAYSFYSHKLYAEACAISEPLCQHLGLVKPGTYPEVPPEKLHRCFRLQVESLKKLGKQAQGCKMVILWLAALQPCSPEHMAEPVTFWVRVKMDAARAGDKELQLKTLRDSLSGWDPETLALLLREELQAYKAVRADTGQERFNIICDLLELSPEETPAGAWARATHLVELAQVLCYHDFTQQTNCSALDAIREALQLLDSVRPEAQARDQLLDDKAQALLWLYICTLEAKIQEGIERDRRAQAPGNLEEFEVNDLNYEDKLQEDRFLYSNIAFNLAADAAQSKCLDQALALWKELLTKGQAPAVRCLQQTAASLQILAALYQLVAKPMQALEVLLLLRIVSERLKDHSKAAGSSCHITQLLLTLGCPSYAQLHLEEAASSLKHLDQTTDTYLLLSLTCDLLRSQLYWTHQKVTKGVSLLLSVLRDPALQKSSKAWYLLRVQVLQLVAAYLSLPSNNLSHSLWEQLCAQGWQTPEIALIDSHKLLRSIILLLMGSDILSTQKAAVETSFLDYGENLVQKWQVLSEVLSCSEKLVCHLGRLGSVSEAKAFCLEALKLTTKLQIPRQCALFLVLKGELELARNDIDLCQSDLQQVLFLLESCTEFGGVTQHLDSVKKVHLQKGKQQAQVPCPPQLPEEELFLRGPALELVATVAKEPGPIAPSTNSAPVLKTKPQPIPNFLSHSPTCDCSLCASPVLTAVCLRWVLVTAGVRLAMGHQAQGLDLLQVVLKGCPEAAERLTQALQASLNHKTPPSLVPSLLDEILAQAYTLLALEGLNQPSNESLQKVLQSGLKFVAARIPHLEPWRASLLLIWALTKLGGLSCCTTQLFASSWGWQPPLIKSVPGSEPSKTQGQKRSGRGRQKLASAPLSLNNTSQKGLEGRGLPCTPKPPDRIRQAGPHVPFTVFEEVCPTESKPEVPQAPRVQQRVQTRLKVNFSDDSDLEDPVSAEAWLAEEPKRRGTASRGRGRARKGLSLKTDAVVAPGSAPGNPGLNGRSRRAKKVASRHCEERRPQRASDQARPGPRIMETIPEEELTDNWRKMSFRILEGSDGEDSASGGKTPAPGPEAASGEWRLLELDSSKKKLPSPCPDKESDKDLGPRLQLPSAPVATGLSTLDSICDSLSVAFRGISHCPPSGLYAHLCRFLALCLGHRDPYATAFLVTESVSITCRHQLLTHLHRQLSKAQKHRGSLEIADQLQGLSLQEMPGDVPLARIQRLFSFRALESGHFPQPEKESFQERLALIPSGVTVCVLALATLQPGTVGNTLLLTRLEKDSPPVSVQIPTGQNKLHLRSVLNEFDAIQKAQKENSSCTDKREWWTGRLALDHRMEVLIASLEKSVLGCWKGLLLPSSEEPGPAQEASRLQELLQDCGWKYPDRTLLKIMLSGAGALTPQDIQALAYGLCPTQPERAQELLNEAVGRLQGLTVPSNSHLVLVLDKDLQKLPWESMPSLQALPVTRLPSFRFLLSYSIIKEYGASPVLSQGVDPRSTFYVLNPHNNLSSTEEQFRANFSSEAGWRGVVGEVPRPEQVQEALTKHDLYIYAGHGAGARFLDGQAVLRLSCRAVALLFGCSSAALAVHGNLEGAGIVLKYIMAGCPLFLGNLWDVTDRDIDRYTEALLQGWLGAGPGAPLLYYVNQARQAPRLKYLIGAAPIAYGLPVSLR

SEQ ID NO: 7 - protein, 2118 aa, Mus musculus
MRNFKGVNFATLLCSKEETQQLLPDLKEFLSRSRTDFPSSRTDAERRQICDTILRACTQQLTAKLDCPGHLRSILDLAELACDGYLLSTPQRPPLYLERILFILLRNGSTQGSPDTVLRLAQPLHACLVQNSGEAAPQDYEAVTRGSFSLFWKGAEALLERRAAFSTRLNALSFLVLLEDGSVPCEVPHFASPTACRLVAAYQLYDATGQGLDEADADFLYEVLSRHLIRVLVGEGGSSPGPLSPQRALCLLEITLEHCRRLCWNHHHRQAARAVERARNHLEKTSVAPSLQLCQMGVELLEAVEERPGAVAQLLRKAAAVLINSIEAPSPPLRALYDSCQFFLSGLERGIRRHCGLDAILSLFAFLGGYSSLVRHLREVSEASSKQQQCLLQMHFQGFHLFTGIVYDFAQGCQATELAQLVDGCRSAAVWMLEALEGLSGGELADYLSMTASYTSNLAYSFFSQKLYEEACVISEPVCQHLGSATSGACPEVPPEKLHRCFRLHVESLKKLGKQAQGCKMVTLWLAALKPYSLEHMVEPVTFWVRVKMDASRAGDKELQLQTLRDSLSCWDPETQSLLLREELRAYKSVRADTGQERFNIICDLLELSPEETAAGAWARATYLVELAQVLCYHNFTQQTNCSALDAVQEALQLLESVSPEAQEQDRLLDDKAQALLWLYICTLEAKMQEGIERDRRAQAPSNLEEFEVNDLNYEDKLQEDRFLYSSIAFNLAADAAQSKCLDQALTLWKEVLTKGRAPAVRCLQQTAASLQILAAVYQLVAKPLQALETLLLLQIVSKRLQDHAKAASSSCQLTQLLLNLGCPSYAQLYLEEAESSLRSLDQTSDACQLLSLTCALLGSQLCWACQKVTAGVSLLLSVLRDPALQKSSKAWYLLRVQALQVLAFYLSLSSNLLSSALREQLWDQGWQTPETALIDAHKLLRSIIILLMGSDVLSIQKAATESPFLDYGENLVQKWQVLTEVLTCSERLVGRLGRLGNVSEAKAFCLEALKLTTKLQIPRQCALFLVLKGELELARGDIDLCQSDLQQVLFLLESSTEFGVVTQHPDSVKKVHTQKGKHKAQGPCFPPLSEEEPFLKGPALELVDTVLNEPGPIQSSVNSSPVLKTKPPPNPGFLSHLPSCDCLLCASPALSAVCLRWVLVTAGVRLATGHKAQGLDLLQAVLTRCPAATKRFTQSLQASLNHRTTPSCVPSLFDEIMAQVYTHLALEFLNQTSEKSLGKVLASGLKFVATRIQSLEIWRAHLLLVQALAKLAHFSCCTSELFASSWGWHPPLVKSLPVLEPAKIRRQKCSGRGRRRIASVPPPLHNSSQKGLEEEGPPCTPKPPGRARQAGPRVPFTIFEEVHPTKSKLQVPLAPRVHRRAQTRLKVIFSDDSDLEDLVSADTQLVEEPKRRGTASRTRGQTRKGRSLKTDAVVAIESTPGHSSVSGRTRRARKVASRNCEEESPKAPLCVWASQGPEIMRSIPEEEPVDNHLEKSFEILRGSDGEDSASGEKAAAADTGLPVGECEVLRRDSSKAERPVLYSDTEANSDPSPWLPPFSVPAPIDLSTLDSISDSLSIAFRGVSHCPPSGLYAHLCRFLALCLGHRDPYATAFLVAESISITCRHQLLTHLHRQLSKAQKQQESPELAEHLQRLDLKERPGGVPLARIQRLFSFKALGSGCFPQAEKESFQERLALIPSGVTVCVLALATLQPGTLSNTLLLTRLEKDNPPITVKIPTAQNKLPLSAVLKEFDAIQKDQKENSSCTEKRVWWTGRLALDQRMEALITALEEQVLGCWRGLLLPCSADPSLAQEASKLQELLRECGWEYPDSTLLKVILSGARILTSQDVQALACGLCPAQPDRAQVLLSEAVGQVQSQEAPRSQHLVLVLDKDLQKLPWESTPILQAQPVTRLPSFRFLLSYTVTKEAGASSVLSQGVDPQNTFYVLNPHSNLSSTEERFRASFSSETGWKGVIGEVPSLDQVQAALTERDLYIYAGHGAGARFLDGQAVLRLSCRAVALLFGCSSAALAVHGNLEGAGIVLKYIMAGCPLFLGNLWDVTDRDIDRYTEALLQGWLGAGPGAPFLYYASQARQAPRLKYLIGAAPVAYGLPISLQTP

SEQ ID NO: 8 - protein, 1262 aa, Caenorhabditis elegans [sequence omitted]

SEQ ID NO: 9 - protein, 1630 aa, Saccharomyces cerevisiae
MMVKQEEPLNEISPNTPMTSKSYLLNDTLSKVHHSGQTRPLTSVLSGDASSNSIGILAMHNNIIRDFTKIASNNIDLAIEDITTVDHSLNSIYSLLKSHHMWGHINSTVKQHLMIIVKLINNNALGLASSEIIFLFNETNLFQAHSLKNILLADFSTWNDYYLSNLKILALQIILKRKLVDEYLPHILELFSHDKRYLLKDPNLKAHALTKIVLSFFSVTTSCKVLFGLKFLQYIKQFKLPFKKFISNITVECFSKNLLHKNYLEMGPNKIYLNSFYLSYSMLYDGLDKIMLLDILSYEETTEVQRAIKSKKEFNEYCNMSENRLLWSCISVDDLNVILENATNFLQNKGKHISATLKCLVCLWSTIRLEGLPKNKDILRQFDCTVIYINSNIKSINDESAAALLSELLGVLSEICIDYKEPKRLSNIISVLFNASVLFKSHSFLLKTANLEISNVLISNDSKTSHRTILKFEKFISSAQSAQKKIEIFSCLFNVYCMLRNDTLSFVFDFCQNAFIHCFTRLKITKFIEFSNSSEIMLSVLYGNSSIENIPSENWSQLSRMIFCSLRGIFDLDPLELNNTFDKLHLLNKYELLIRIVYLLNLDMSKHLTTNLSKITKLYINKWLQKSDEKAERISSFEMDFVKMLLCYLNFNNFDKLSIELSLCIKSKEKYYSSIVPYADNYLLEAYLSLYMIDDALMMKNQLQKTMNLSTAKIEQALLHASSLINVHLWDSDLTAFQIYFGKTLPAMKPELFDINNDHNLPMSLYIKVILLNIKIFNESAKLNIKAGNVISAVIDCRKAQNLALSLLKKKNKLSQGSRLALLKSLSFSFFQLIKIHIRIGSARDCEFYSKELSRIISDLEEPIIVYRCLHFLHRYYMITEQTCLQNITLGKANKAFDYLDAEADITSLTMFLYDNKEFVKLEQSLVLYFGDQLEKTFLPNLWKLHLGKDIDDSICLSEYMPKNVINRVHNMWQKVMSQLEEDPFFKGMFESTLGIPSSLPVIPSTMPNNILKTPSKHSTGLKLCDSPRSSSMTPRGKNIRQKFDRIAAISKLKQMKELLESLKLDTLDNHELSKISSLSSLTLTILSNITSIHNAESSLITNFSLTDLPRHMPLLFDKVLNNIDNKNYREFRVSSLIAPNNISTITESIRVSAAQKDLMESNLNINVITIDFCPITGNLLLSKLEPRRKRRTHLRLPLIRSNSRDLDEVHLSFPEATKKLLSIINESNQTTSVEVTNKIKTREERKSWWTTRYDLDKRMQQLLNNIENSWFNGVQGFFSPEVVDNSLFEKFKDKFYEILHQNLPSRKLYGNPAMFIKVEDWVIELFLKLNPQEIDFLSKMEDLIYFVLDILLFHGEENAYDEIDFSMLHVQLEEQIKKYRATMTTNSIFHTFLVVSSSCHLFPWECLSFLKDLSITRVPSYVCLNKLLSRFHYQLPLQVTIEDNISMILNPNGDLSRTESKFKGMFQKIIDAKPSSQLVMNEKPEEETLLKMLQNSNLFVYIGHGGGEQYVRSKEIKKCTKIAPSFLLGCSSAAMKYYGKLEPTGTIYTYLLGGCPMVLGNLWDVTDKDIDKFSEELFEKMGFRCNTDDLNGNSLSVSYAVSKSRGVCHLRYLNGAAPVIYGLPIKFVS

SEQ ID NO: 10 - protein, 1828 aa, Schizosaccharomyces pombe
MSTRSIVTSKVSWTPEKFISALSYPEHCSITLVKRLKASVKLKDLKQNISRDAPSWTFEHLFVAFKCAVSNLAKQWAELSTTDKEKTRRMFCTPSRLNTAHRPEVFYLLECCTYILEQMQVVTKNTSHLYDCIRSGVSICNRLLDMEIFEPAISLLMKTHKNLIILLTYRDHDAIPTATLLNPTLDVSEIQLESCLFVPMVPASYFLNIGTIVVTFQLNVLRCLSLSQINGLSLNTINNLQSEDGPFQWIERSFPSQVQLANSRREILARLLTRFSMIQNNALQSFKLLILSIALWLNILSSQRADDKEFDVNQLETRILQLFSKVVQLCKSEDIEGSILNKDMTQLHHLLENLSKESRLHILLQLSQLYYKYNDFQLSAAYVIRGYSLSFEDISFKLKFLLFSFRLSIHDNSICFPFNLIQELSSLQQLFVENALPYSEALHLLDSIERSFRLFNDSTVFDDTVFALNISEILSWILSSVVRDILVEDELLNLQLKIRKFLMFTFHIIRSFSELTKFQSSLEGCLNLAAYYEDAEFPQKLSNHLYNLCVKSSNVNYARECISLSIKIAVSHKLTNDETYLLKILKNFQLRYHDSLQLQEKCDVLHTTFNQLDLYVGTTSVGKSSVLDNILKRIFNSLTSINDSNIEKLLESISYSLLKLFFKCANEGSRYNASAALSFKLSLMLHEKEEVLLLKTNVSCVLANHGYNDIKFEEMVLCVIKGDQNLLEHNSNNNAKLALNESLLCSWENLLCYRRAEDDSRILTIIESWTIFISRFSSVISRCSFTDFEINSILNFFFCFLHTVEPSGKLTFELAFLEIFYELFNCLLHLQFSKYLVIIGTLLSDKYMTLGFSGKAHLFYTKCYSYLRQCKSSPFINFWNVSYGKYLILTGNTDKGILQLKKYSLSSEEDFNSNGLSRTVSLNLLLYERIQLSDALFQLGYTTVSLGFIMQNLKVIKGLFSKSSKEHFNGGKYITWRLFAVSAHSNVCAARIYEHMGQAREAEFFYRQACSISEKMPFSCFSATFQLRLCSLLTRAGKLEKGEKILFDLTEAMKSTDTYHKLLWNYGAAEVCATKSELDGAICHYSECVKLLEIIKSEYYLFFNRNREKSLTKGIKRLSLSSQPTFVTESNTTEFDDWSILQNTAANLLRLISMFELKRGNLEIAKALMTDSTKCSIASFFNIVSANILKSKLIVCEADSTLFGDPVLRTLPDSVISLPGISHKFQKNQSKTKALGENTGFRKGSKRLDYLRERLKINLQNVRLSCEIIFSNAYERSSVCVCREVNELISYSTIMQSALTTIGETTDVDSSSASFFLEIPKALGFHRRREAQKFRNQHKELHFSSLEQILNSRLSIPDVRTFQDNFIDSLPSIWNVVSITINNSGEDLFISKIRKGHSPLIFRLPLQRHNSRDADEEILVFTKAQTELFRIISKSNQMAQNGKHYTRREDKETWWKERRHLDQCLQQLLENIEISWLGGFKGIFNPHKIDTSLFAKFSSQFQNIIAKNFNMDKKTPVPTLSPEILELFITLGKPGYEGYEQLLEDLIYFILDIFQFRGLHFAYDEIDTDQLSMDLQDALNAYFNNYVSEENRSHTVLVLDKSVHQFPWESLPCLNRQSVSRVPSLSILRDILSQSFVVNGEYVEVRKEAGSYILNPSLDLKHTQEMFEHKLVEGGWKGLIASQPSNRDFIKMLSGNDFFLYFGHGGGEQYTTSYDLATLKRCAVTILMGCSSGALYECGSFEPWGTPLDYLSAGCPTLVANLWDVTDKDIDRFSLKMLESWGLFENKAPFVNSTSICTAVSESRSCCHLRYLNGAAPVIYGIPAYIIP

SEQ ID NO: 11 - protein, 199 aa, Mus musculus
MATLIFVDKDNEEPGRRLASKDGLKLGTGVKALDGKLQVSTPRVGKVFNAPAVPKASRKALGTVNRVAEKPMKTGKPLQPKQPTLTGKKITEKSTKTQSSVPAPDDAYPEIEKFFPFNPLDFESFDLPEEHQISLLPLNGVPLMTLNEERGLEKLLHLGPPSPLKTPFLSWESDPLYSPPSALSTLDVELPPVCYDADI

SEQ ID NO: 12 - protein, 244 aa, Caenorhabditis elegans
MEDLNFEERGSTQIPASLQQHFSAKLGRQNELEKTPSRGGLGLVVNSSKTPGGKSLQSLASACKVPPSTKKNTIPIAFECYEDETDDQIADVATIKKTEKHPCSPIDTANRCETFDSLAADIEDDMLNLEDQDVVLSEDRPYGDVIDPAESEAEALAELGVEEWDSYPPIDPASRIGDDFNYVLRTEDFAEEGDVKLEETRHRTVIADIDEVKMSKAERNELFSMLADDLDSYDLLAEEANLPL

SEQ ID NO: 13 - protein, 373 aa, Saccharomyces cerevisiae
MMPANEDKENNIVYTGNESSGINFPQTPAHLLKRSHSNILKPPVRLDQLKRDANSNNGNTLKYIQGGKEVSPTKRLHTHAQQQGRLPLAAKDNNRSKSFIFPETSNQSKDADLPQLQNTLSIRKNDQLRKLSQISRSRSRANHNDLLSNSRKLQKYGSVLGYNALPKMKSLVLKDLADSGKNEESSDDDEGNEDSESKLGKKLQSALLKQDSSDGENELNGGLGLFNEQGGLQQLIKNSTKNEQKTKNDKSDKTDDYDIEIAPQRQEPLPYVPEGYSPFQQDDIEKLKTFNSPYKLDLEDEDDTPDKVDLLPLEQIDEEGEKDETECITRNQEEGAALPLLSKNFKEVAAVPTMELVYSEEGLDPEELEDLVT

SEQ ID NO: 14 - protein, 301 aa, Schizosaccharomyces pombe
MLPRTMFSYGKENAFPVTPISNRNGTKGAGSKRAPLGSTKQSNAPSSVTVPRTVLGGKSTNISKFISAPSTKKMSPMDISMDSPTILEPNSQGISRSAVQERSKRLSASPRRSSLTDTPLPNELEEDIEYMPPPVHLDPIQSLGFDDVAIDCETLDPWPSMQNKATSVTIRNTPASDFHVYKEFSDDDPIQFPLLSVDGDSPLTEKDTNLTTPATLKASDQQRKVLEKPSVSKQSSSRTRLSTVYRTKLASGKSIPRPLSHKLTRPRVTASGNSRRRPLSRSIHSLSSSRIDFSSLDTGLL

SEQ ID NO: 15 - protein, 5 aa, Artificial Sequence
  Description of Artificial Sequence Synthetic peptide
GGGGS

SEQ ID NO: 16 - protein, 10 aa, Artificial Sequence
  Description of Artificial Sequence Synthetic peptide
GGGGSGGGGS

SEQ ID NO: 17 - protein, 15 aa, Artificial Sequence
  Description of Artificial Sequence Synthetic peptide
GGGGSGGGGSGGGGS

SEQ ID NO: 18 - protein, 20 aa, Artificial Sequence
  Description of Artificial Sequence Synthetic peptide
GGGGSGGGGSGGGGSGGGGS

SEQ ID NO: 19 - protein, 6 aa, Artificial Sequence
  Description of Artificial Sequence Synthetic peptide
GGGGGG

SEQ ID NO: 20 - protein, 8 aa, Artificial Sequence
  Description of Artificial Sequence Synthetic peptide
GGGGGGGG

SEQ ID NO: 21 - protein, 15 aa, Artificial Sequence
  Description of Artificial Sequence Synthetic peptide
GGSGGSGGGSGGGSG

SEQ ID NO: 22 - protein, 6 aa, Artificial Sequence
  Description of Artificial Sequence Synthetic 6xHis tag
HHHHHH

SEQ ID NO: 23 - protein, 8 aa, Artificial Sequence
  Description of Artificial Sequence Synthetic peptide
DYKDDDDK

SEQ ID NO: 24 - protein, 11 aa, Unknown
  Description of Unknown unfoldase recognition site sequence
TNTAKILNFGR

SEQ ID NO: 25 - protein, 10 aa, Artificial Sequence
  Description of Artificial Sequence Synthetic peptide
DDREIMREGS

SEQ ID NO: 26 - protein, 7 aa, Homo sapiens
EIEKFFP

SEQ ID NO: 27 - protein, 4 aa, Homo sapiens
EIMR

SEQ ID NO: 28 - protein, 4 aa, Homo sapiens
EPSR

SEQ ID NO: 29 - protein, 21 aa, Unknown
  Description of Unknown Scc1 sequence
PPALSEAGVMLPEQPAHDDMD

SEQ ID NO: 30 - protein, 30 aa, Homo sapiens
YPEIEKFFPFNPLDFESFDLPEEHQIAHLP

SEQ ID NO: 31 - protein, 30 aa, Mus musculus
YPEIEKFFPFNPLDFESFDLPEEHQISLLP

SEQ ID NO: 32 - protein, 30 aa, Gallus gallus
YPEIENLFPSDPLDLESFDVPEEHRLSNIN

SEQ ID NO: 33 - protein, 30 aa, Danio rerio
LPEIEKFFPYNPSEFECYSVPDEVYLSRFS

SEQ ID NO: 34 - protein, 28 aa, Caenorhabditis elegans
AADIEDDMLNLEDQDVVLSEDRPYGDVI

SEQ ID NO: 35 - protein, 27 aa, Saccharomyces cerevisiae
DYDIEIAPQRQEPLPYVPEGYSPFQQD

SEQ ID NO: 36 - protein, 31 aa, Chaetomium thermophilum
VEEVEYAPPKPKEMPYESDVFPEGVLTYEGL

SEQ ID NO: 37 - protein, 30 aa, Schizosaccharomyces pombe
EEDIEYMPPPVHLDPIQSLGFDDVAIDCET